SEQUENCE LISTING (108 sequences)

SEQ ID NO: 1 - DNA, 1389 bp, Mus musculus
  CDS (1)..(1389)
atggcttgggtgtggaccttgctattcctgatggcagctgcccaaagtatccaagcacagagccacttggtgcagtctggacctgagctgaagaagcctggagagacagtcaggatctcatgcaaggcttctggttataccttcacagacttttcaatgcagtgggtgaagcaggctccaggaaagggtttaaagtggatgggctggataaacactgcgactggtgagccaacatatgcagatggcttcaagggacgctttgtcttctctctggaaacctctgccaacactgcctatttgcagatcacaaacctcaagagggaagacacggctacatatctctgtgtcagtggtggggactactggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 2 - protein, 462 aa, Mus musculus
MAWVWTLLFLMAAAQSIQAQSHLVQSGPELKKPGETVRISCKASGYTFTDFSMQWVKQAPGKGLKWMGWINTATGEPTYADGFKGRFVFSLETSANTAYLQITNLKREDTATYLCVSGGDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 5 aa, Mus musculus
DFSMQ

SEQ ID NO: 4 - protein, 17 aa, Mus musculus
WINTATGEPTYADGFKG

SEQ ID NO: 5 - protein, 4 aa, Mus musculus
GGDY

SEQ ID NO: 6 - DNA, 717 bp, Mus musculus
  CDS (1)..(717)
atgagtcctgcccagttcctgcttctgttagtgctctggaatcgggaaaccaacggtgatattgtgatgacccagactcctctcactttgtcggttacccttggacaaccagcctccatctcttgcaagtcaagtcagagcctcttagatactgatggaaagacatatttgaattggttgttacagaggccaggccagtctctcgtccgcctgatttatctggtatctaaactggactctggagtccctgacaggttcagtggcagtggatcagggacagatttcaccctgaaaatcagcagagtgggggctgaggatttggggatttattattgctggcaaggtacacattttcctcccacgttcggaggggggaccaggctggaaatgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 7 - protein, 238 aa, Mus musculus
MSPAQFLLLLVLWNRETNGDIVMTQTPLTLSVTLGQPASISCKSSQSLLDTDGKTYLNWLLQRPGQSLVRLIYLVSKLDSGVPDRFSGSGSGTDFTLKISRVGAEDLGIYYCWQGTHFPPTFGGGTRLEMKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 16 aa, Mus musculus
KSSQSLLDTDGKTYLN

SEQ ID NO: 9 - protein, 7 aa, Mus musculus
LVSKLDS

SEQ ID NO: 10 - protein, 9 aa, Mus musculus
WQGTHFPPT

SEQ ID NO: 11 - DNA, 1398 bp, Mus musculus
  CDS (1)..(1398)
atggcttgggtgtggaccttgctattcctgatggcagctgcccaaagtatccaagcacagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggcttaacactgagactggtgagccaacatatacagatgacttcaagggacggtttgccttctctttggaaacctctgccagcgctgcctatttgcagatcacgaacctcagaaatgaggacacggctacatatttctgtgcttctcacccctatggtatgtcctactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 12 - protein, 465 aa, Mus musculus
MAWVWTLLFLMAAAQSIQAQIQLVQSGPELKKPGETVKISCKASGYTFTDYSMHWVKQAPGKGLKWMGWLNTETGEPTYTDDFKGRFAFSLETSASAAYLQITNLRNEDTATYFCASHPYGMSYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 5 aa, Mus musculus
DYSMH

SEQ ID NO: 14 - protein, 17 aa, Mus musculus
WLNTETGEPTYTDDFKG

SEQ ID NO: 15 - protein, 7 aa, Mus musculus
HPYGMSY

SEQ ID NO: 16 - DNA, 717 bp, Mus musculus
  CDS (1)..(717)
atgagtcctgcccagttcctgtttctgttagtgctctggattcgggaaagcaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtccacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 17 - protein, 238 aa, Mus musculus
MSPAQFLFLLVLWIRESNGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPSTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 18 - protein, 16 aa, Mus musculus
KSSQSLLDSDGKTYLN

SEQ ID NO: 19 - protein, 7 aa, Mus musculus
LVSKLDS

SEQ ID NO: 20 - protein, 9 aa, Mus musculus
WQGTHFPST

SEQ ID NO: 21 - DNA, 1395 bp, Mus musculus
  CDS (1)..(1395)
atggattggctgaggaacttgctattcctgatggcagctgcccaaagtgcccaagcacagatccagttggtgcagtctggacctgagctgaagaagcctggagagacagtcaagatctcctgcaaggcttctgggtataccttcacagactatggagtgaactgggtgaagcaggctccaggaaagggtttaaagtggatgggctggataaacacctacactggagagccaacctatgctgatgacttcaagggacggtttgacttctttttggaaacttctgccagcactgcctatttgcagatcaacaacctcaaaaatgaggacatggctacatatttctgtagtagctacgagtactttgactactggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 22 - protein, 464 aa, Mus musculus
MDWLRNLLFLMAAAQSAQAQIQLVQSGPELKKPGETVKISCKASGYTFTDYGVNWVKQAPGKGLKWMGWINTYTGEPTYADDFKGRFDFFLETSASTAYLQINNLKNEDMATYFCSSYEYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 23 - protein, 5 aa, Mus musculus
DYGVN

SEQ ID NO: 24 - protein, 17 aa, Mus musculus
WINTYTGEPTYADDFKG

SEQ ID NO: 25 - protein, 6 aa, Mus musculus
YEYFDY

SEQ ID NO: 26 - DNA, 723 bp, Mus musculus
  CDS (1)..(723)
atgagtcctgcccagttcctgtttctgttagtgctctggattcgggtttcagaaaccaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctgggaaggtacacattttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 27 - protein, 240 aa, Mus musculus
MSPAQFLFLLVLWIRVSETNGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWEGTHFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 28 - protein, 16 aa, Mus musculus
KSSQSLLDSDGKTYLN

SEQ ID NO: 29 - protein, 7 aa, Mus musculus
LVSKLDS

SEQ ID NO: 30 - protein, 9 aa, Mus musculus
WEGTHFPYT

SEQ ID NO: 31 - DNA, 1398 bp, Mus musculus
  CDS (1)..(1398)
atggcttgggtgtggaccttgttattcctgatggcagctgcccaaagtatccaagcacagatccagttggtgcagtctggacctgagctgaagaagcctggagagacagtcgaaatctcctgcaaggcttctggttataccttcacagactattcaatgcactgggtgaagcaggctccaggaaagggtttaaagtggatggcctggataaacactgcgactggtgagccaatgtatgtagatgacttcgagggacggtttgacttgtctttagaagcctctgtcagcactgtctatttgcggatcagcaacctcaaaaatgaggacacggcaacatatttctgcagtagttatcccgattctatggactattggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 32 - protein, 465 aa, Mus musculus
MAWVWTLLFLMAAAQSIQAQIQLVQSGPELKKPGETVEISCKASGYTFTDYSMHWVKQAPGKGLKWMAWINTATGEPMYVDDFEGRFDLSLEASVSTVYLRISNLKNEDTATYFCSSYPDSMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 5 aa, Mus musculus
DYSMH

SEQ ID NO: 34 - protein, 17 aa, Mus musculus
WINTATGEPMYVDDFEG

SEQ ID NO: 35 - protein, 7 aa, Mus musculus
YPDSMDY

SEQ ID NO: 36 - DNA, 717 bp, Mus musculus
  CDS (1)..(717)
atgagtcctgcccagttcctgtttctgttagtgctctggattcgggagatcaacggtgctattgtgatgacccagactccactcactttgtcggttaccattggacaaccagcctccatctcttgcaagtcaagtcagagcctcttagatagtgatggaaggacatatttgaattggttgtttcagaggccaggccagtctccaaagcgcctaatctatctggtgtctaatctggactctggagtccctgacaggttcactggcagtggatcagggacagatttcacactgaaaatcagcagagtggaggctgaggatttgggatttttttattgctggcaaggaacacattttcctccgacgttcggtgggggcaccaagctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 37 - protein, 238 aa, Mus musculus
MSPAQFLFLLVLWIREINGAIVMTQTPLTLSVTIGQPASISCKSSQSLLDSDGRTYLNWLFQRPGQSPKRLIYLVSNLDSGVPDRFTGSGSGTDFTLKISRVEAEDLGFFYCWQGTHFPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - protein, 16 aa, Mus musculus
KSSQSLLDSDGRTYLN

SEQ ID NO: 39 - protein, 7 aa, Mus musculus
LVSNLDS

SEQ ID NO: 40 - protein, 9 aa, Mus musculus
WQGTHFPPT

SEQ ID NO: 41 - DNA, 1389 bp, Mus musculus
  CDS (1)..(1389)
atggcttgggtgtggaccttgctattcctgatggcagctgcccaaagtatccaagcacagatccagttggtgcaatctggacctgcgctgaagaagcctggagagacagtcaagatctcctgcaaggcttctggttataccttcacagactattcaatgcagtgggtgaagcaggctccaggaaagggtttaaagtggatgggctggataaatactgagactggtgagccaacatatgcagatgacttcaagggacggtttgccttctctttggaaacctctggcagcactgcctatttgcagatcaacaacctcaaaaatgaggacacggcttcatatttctgtactagtcagggtgcctactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 42 - protein, 462 aa, Mus musculus
MAWVWTLLFLMAAAQSIQAQIQLVQSGPALKKPGETVKISCKASGYTFTDYSMQWVKQAPGKGLKWMGWINTETGEPTYADDFKGRFAFSLETSGSTAYLQINNLKNEDTASYFCTSQGAYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 43 - protein, 5 aa, Mus musculus
DYSMQ

SEQ ID NO: 44 - protein, 17 aa, Mus musculus
WINTETGEPTYADDFKG

SEQ ID NO: 45 - protein, 4 aa, Mus musculus
QGAY

SEQ ID NO: 46 - DNA, 714 bp, Mus musculus
  CDS (1)..(714)
atgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacatcttcccacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 47 - protein, 237 aa, Mus musculus
MSPAQFLFLLVLWIRETNGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHLPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 48 - protein, 16 aa, Mus musculus
KSSQSLLDSDGKTYLN

SEQ ID NO: 49 - protein, 7 aa, Mus musculus
LVSKLDS

SEQ ID NO: 50 - protein, 8 aa, Mus musculus
WQGTHLPT

SEQ ID NO: 51 - DNA, 1395 bp, Mus musculus
  CDS (1)..(1395)
atggattggctgaggaacttgctattcctgatggcagctgcccaaagtgcccaagcacagatccagttggtgcagtctggacctgagctgaagaagcctggagagacagtcaagatctcctgcaaggcttctgggtataccttcacagactatggagtgaactgggtgaagcaggctccaggaaagggtttaaagtgggtgggctggataaacacctacactggagagccaacatatgctgatgacttcaagggacggtttgccttctctttggaaacctctgccagtactgcctatttgcagatcaacaacctcaaaaatgaggacatggccacatatttctgtagtagttatgaatactttgactactggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 52 - protein, 464 aa, Mus musculus
MDWLRNLLFLMAAAQSAQAQIQLVQSGPELKKPGETVKISCKASGYTFTDYGVNWVKQAPGKGLKWVGWINTYTGEPTYADDFKGRFAFSLETSASTAYLQINNLKNEDMATYFCSSYEYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 53 - protein, 5 aa, Mus musculus
DYGVN

SEQ ID NO: 54 - protein, 10 aa, Mus musculus
WINTYTGEPT

SEQ ID NO: 55 - protein, 8 aa, Mus musculus
SSYEYFDY

SEQ ID NO: 56 - DNA, 723 bp, Mus musculus
  CDS (1)..(723)
atgagtcctgcccagttcctgtttctgttagtgctctggattcgggtttcagaaaccaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctgggaaggtacacattttccgtacacgttcggaggggggaccaaactggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 57 - protein, 240 aa, Mus musculus; identical to SEQ ID NO: 27

SEQ ID NO: 58 - protein, 16 aa, Mus musculus
KSSQSLLDSDGKTYLN

SEQ ID NO: 59 - protein, 7 aa, Mus musculus
LVSKLDS

SEQ ID NO: 60 - protein, 9 aa, Mus musculus
WEGTHFPYT

SEQ ID NO: 61 - DNA, 1407 bp, Mus musculus
  CDS (1)..(1407)
atggcttgggtgtggaccttgctattcctgatggcagctgcccaaagtacccaagcacagaaccacttggtgcagtctggacctgaactgaagaagcctggagagacggtcaagatctcctgcaaggcctctggttatatcttcacagactattcaatgcagtgggtgaagcaggctccaggaaagggtttaaagtggatgggctggataaacactgagactgctgaaccaacatatgcagatgacttcaagggacggtttgacttctctttggaaacttctgtcagtactgcctatttacagatcaacaacctcaaaaatgaagacacggctacatatttctgttgtagtgggggggactactggggtcaaggaaccccagtcaccgtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatga

SEQ ID NO: 62 - protein, 468 aa, Mus musculus
MAWVWTLLFLMAAAQSTQAQNHLVQSGPELKKPGETVKISCKASGYIFTDYSMQWVKQAPGKGLKWMGWINTETAEPTYADDFKGRFDFSLETSVSTAYLQINNLKNEDTATYFCCSGGDYWGQGTPVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 63 - protein, 13 aa, Mus musculus
KASGYIFTDYSMQ

SEQ ID NO: 64 - protein, 10 aa, Mus musculus
WINTETAEPT

SEQ ID NO: 65 - protein, 5 aa, Mus musculus
SGGDY

SEQ ID NO: 66 - DNA, 717 bp, Mus musculus
  CDS (1)..(717)
atgagtcctgcccagttcctgtttctgttagtgctctggattcggaaaaccaacggtgatgttgtgatgacccagactccactcactttgtcggttacccttggacaaccagccgccatctcttgcacgtcaagtcagagcctcttagatactgatggaaagacatatttgaattggttgttacagaggccaggccagtctccaaagcgcctgatctatctggtgtctaaactggactctggagtccctgacaggttcactggcagtggatcagggacagatttcacactgaaaatcagcagagtggaggctgacgatttgggagtttattattgctggcaaggttcacattttccgaccacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 67 - protein, 238 aa, Mus musculus
MSPAQFLFLLVLWIRKTNGDVVMTQTPLTLSVTLGQPAAISCTSSQSLLDTDGKTYLNWLLQRPGQSPKRLIYLVSKLDSGVPDRFTGSGSGTDFTLKISRVEADDLGVYYCWQGSHFPTTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 68 - protein, 16 aa, Mus musculus
TSSQSLLDTDGKTYLN

SEQ ID NO: 69 - protein, 7 aa, Mus musculus
LVSKLDS

SEQ ID NO: 70 - protein, 9 aa, Mus musculus
WQGSHFPTT

SEQ ID NO: 71 - DNA, 1389 bp, Mus musculus
  CDS (1)..(1389)
atggcttgggtgtggaccttgctattcctgatggcagctgcccaaagtatccaagcacagatccagttggtgcagtctggacctgagctgaagaagcctggagagacagtcaagatctcctgcaaggcttctggttacaccttcacagactattcaatgcactgggtgaagcaggctccaggaaagggtttaaaatggatgggctggataaacactgagactggtgagccaacatatacagatgacttcaagggacgatttggcttctctttggaaacctctgccaacactgcctacttgcagatcaacaacctcaaaaatgaggacacggctacatatttctgttgtagtggtggagactcctggggccaaggctccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 72 - protein, 462 aa, Mus musculus
MAWVWTLLFLMAAAQSIQAQIQLVQSGPELKKPGETVKISCKASGYTFTDYSMHWVKQAPGKGLKWMGWINTETGEPTYTDDFKGRFGFSLETSANTAYLQINNLKNEDTATYFCCSGGDSWGQGSTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 73 - protein, 13 aa, Mus musculus
KASGYTFTDYSMH

SEQ ID NO: 74 - protein, 10 aa, Mus musculus
WINTETGEPT

SEQ ID NO: 75 - protein, 5 aa, Mus musculus
SGGDS

SEQ ID NO: 76 - DNA, 717 bp, Mus musculus
  CDS (1)..(717)
atgagtcctgcccagttcctgtttctgttactgctctggattcgggagaccaacggtgatgttgtcctgacccagactccactcactctgtcggttaccattggacagccagcctccatctcttgcaagtcaagtcagagcctcttagatagtgatggaaagacacgtttgaattggttgttacagaggccaggccagtctccaaagcgcctaatatatctggtgtctaaactggactctggagtccctgacaggttcactggcagtggatcagggacagctttcacactgaaaatcagcagagtggaggctgaggatttgggagtttattattgctggcaaggtacacattttccatccacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 77 - protein, 238 aa, Mus musculus
MSPAQFLFLLLLWIRETNGDVVLTQTPLTLSVTIGQPASISCKSSQSLLDSDGKTRLNWLLQRPGQSPKRLIYLVSKLDSGVPDRFTGSGSGTAFTLKISRVEAEDLGVYYCWQGTHFPSTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 78 - protein, 16 aa, Mus musculus
KSSQSLLDSDGKTRLN

SEQ ID NO: 79 - protein, 7 aa, Mus musculus
LVSKLDS

SEQ ID NO: 80 - protein, 9 aa, Mus musculus
WQGTHFPST

SEQ ID NO: 81 - protein, 24 aa, Artificial Sequence
  synthetic peptide
MQLIKGQTQNFKDAFSGRDSSITR

SEQ ID NO: 82 - protein, 22 aa, Artificial Sequence
  synthetic peptide
KGQTQNFKDAFSGRDSSITRLP

SEQ ID NO: 83 - protein, 461 aa, Mus musculus
MAWVWTLLFLMAAAQSIQAQIQLVQSGPELKKPGETVKISCKASGYTFTDYSMHWVKQAPGKGLKWMGWINTETGEPTYADDFKGRFAFSLETSASTAYLQINNLKNEDTATYFCVSGSNWGQGTTLIVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 84 - protein, 13 aa, Mus musculus
KASGYTFTDYSMH

SEQ ID NO: 85 - protein, 10 aa, Mus musculus
WINTETGEPT

SEQ ID NO: 86 - protein, 10 aa, Mus musculus
WINTETGEPT

SEQ ID NO: 87 - protein, 238 aa, Mus musculus
MSPAQFLFLLVLWIRETNGDVVMTHTPLTLSVTIGQPASISCKSSQSLLDSDGKTYLNWLLQRPGQSPKRLIYLVSKLDSGVPDRFTGSGSGTDFTLKISRVEAEDLGVYYCWQGTHLPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 88 - protein, 17 aa, Mus musculus
CKSSQSLLDSDGKTYLN

SEQ ID NO: 89 - protein, 7 aa, Mus musculus
LVSKLDS

SEQ ID NO: 90 - protein, 10 aa, Mus musculus
WQGTHLPLTF

SEQ ID NO: 91 - protein, 462 aa, Mus musculus
MAWVWTLLFLMAAAQSIQAQIQLVQSGPELKKPGETVKISCKASGYIFTDYSMHWVKQAPGKGLKWMGWINTATGEPTYVDDFKGRFAFSLETSANTAYLQIIYLRNEDTATYFCCSGGDSWGQGSTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 92 - protein, 5 aa, Mus musculus
DYSMH

SEQ ID NO: 93 - protein, 17 aa, Mus musculus
WINTATGEPTYVDDFKG

SEQ ID NO: 94 - protein, 4 aa, Mus musculus
GGDS

SEQ ID NO: 95 - protein, 240 aa, Mus musculus
MSPAQFLFLLVLWIRVSETNADVVLTQTPLTLSVTIGQPASISCKSSQSLLDSDGKTRLNWLLQRPGQSPKRLMYLVSKLDSGVPDRFTGSGSGTAFTLKISRVEAEDLGVYYCWQGTHFPSTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 96 - protein, 16 aa, Mus musculus
KSSQSLLDSDGKTRLN

SEQ ID NO: 97 - protein, 7 aa, Mus musculus
LVSKLDS

SEQ ID NO: 98 - protein, 9 aa, Mus musculus
WQGTHFPST

SEQ ID NO: 99 - protein, 462 aa, Mus musculus
MAWVWTLLFLMAAAQSIQAQIQLVQSGPELKKPGETVKISCKASGYIFTDYSMHWVKQAPGKGLKWMGWINTETGEPTYVDDFKGRFAFSLETSANTAYLQIIYLKNEDTATYFCCSGGDSWGQGSTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 100 - protein, 5 aa, Mus musculus
DYSMH

SEQ ID NO: 101 - protein, 17 aa, Mus musculus
WINTETGEPTYVDDFKG

SEQ ID NO: 102 - protein, 4 aa, Mus musculus
GGDS

SEQ ID NO: 103 - protein, 240 aa, Mus musculus; identical to SEQ ID NO: 95

SEQ ID NO: 104 - protein, 16 aa, Mus musculus
KSSQSLLDSDGKTRLN

SEQ ID NO: 105 - protein, 7 aa, Mus musculus
LVSKLDS

SEQ ID NO: 106 - protein, 9 aa, Mus musculus
WQGTHFPST

SEQ ID NO: 107 - protein, 330 aa, Homo sapiens [sequence omitted]

SEQ ID NO: 108 - protein, 107 aa, Homo sapiens [sequence omitted]

(19) United States
(12) Patent Application Publication
(10) Pub. No.: US 2022/0213187 A1
(43) Pub. Date: Jul. 7, 2022

(54) COMPOSITIONS AND METHODS RELATED TO XCT ANTIBODIES

(71) Applicant: Agilvax Inc., Austin, TX (US)

(72) Inventors: FEDERICA CAVALLO, Torino (IT); AHMAD SALAMEH, Houston, TX (US); JERRI CALDEIRA, Albuquerque, NM (US); FEDERICA PERICLE, El Paso, TX (US)

(21) Appl. No.: 17/609,442

(22) PCT Filed: May 8, 2020
(86) PCT No.: PCT/US20/32121

§ 371 Date: Nov. 8, 2021

Related U.S. Application Data
(60) Provisional application No. 62/845,264, filed on May 8, 2019.
(60) Provisional application No. 62/942,170, filed on Dec. 1, 2019.

(51) Int. Cl. C07K 16/28; A61K 47/68; A61P 35/00

(52) U.S. Cl. CPC C07K 16/28; A61K 2039/505; A61P 35/00; A61K 47/6849

(57) ABSTRACT

Certain embodiments are directed to therapeutic compositions having an xCT specific antibody.

### RELATED APPLICATION

This application claims priority to U.S. Provisional Patent Applications Ser. No. 62/845,264 filed May 8, 2019 and 62/942,170 filed Dec. 1, 2019, each of which is incorporated herein by reference in its entirety.

### BACKGROUND

Many tumors contain phenotypically and functionally heterogeneous cancer cells which can lead to aggressive progression due to the enrichment of cancer stem cells (CSC). CSCs have the unique biological properties necessary for maintenance and spreading of the tumor and can differentiate into cancer cells that compose the tumor bulk through asymmetric division (Magee et al., Cancer Cell 2012, 21(3):283-96). Due to their resistance to traditional radio- and chemo-therapies (Nagano et al., Oncogene 2013, 32(44):5191-8), CSCs represent a reservoir for the relapse, metastatic evolution, and progression of the disease after treatment. Therefore, successful eradication of CSC represents a major barrier towards effective cancer treatments.

The ability of CSC to resist common cytotoxic therapies relies on different mechanisms, including improved detoxification ability. The cystine-glutamate antiporter protein xCT (SLC7A11) regulates cysteine intake, conversion to cysteine and subsequent glutathione synthesis, protecting cells against oxidative and chemical insults via the p38MAPK pathway (Chen et al., Oncogene 2009, 28(4):599-609; Guo et al., Cancer Lett. 2011, 312(1):55-61). Increased tumor xCT expression is a significant predictor for metastatic progression and reduction of patient survival in lung, colorectal, hepatocellular, and breast cancer patients (Briggs et al., Cell 2016, 166(1):126-39; Gyorffy et al., Breast Cancer Res Treat. 2010, 123(3):725-31; Suganao et al., Anticancer Res., 2015, 35:677-82; Ji et al., Oncogene. 2018, 37:5007-19; Cohen et al., Oncotarget, 2017, 8:113373-402; Kinoshita et al., Oncol Rep. 2013, 29:685-9). xCT expression is upregulated in solid tumor stem cells, and several studies show that xCT physically interacts with the well-known stem cell marker, CD44, which is shown to be highly expressed in CSC's for breast, prostate, colon, head and neck, pancreatic, and other solid tumors (Nagano et al., Oncogene 2013, 32(44):5191-8; Hasegawa et al., Oncotarget 2016, 7(11):11756-69; Ishimoto et al., Cancer Cell 2011, 19(3):387-400; Ju et al., Mechanisms and Therapeutic Implications. Theranostics 2016, 6(8):1160-75; Yoshikawa et al., Cancer Res. 2013, 73(6):1855-66). The frequency of xCT expression on a variety of CSC suggests that therapies targeting xCT may be effective for a variety of tumors with high stem cell frequencies.

A direct role for xCT in cancer metastasis was shown by inhibiting xCT function with the small molecule sulfasalazine (SASP), which resulted in significant decreases in metastatic foci in animal models and reductions in the frequency of CSC (Guan et al., Cancer Chemother Pharmacol. 2009, 64(3):463-72; Timmerman et al., Cancer Cell 2013, 24(4):450-65). However, SASP is labile and insoluble under physiological conditions, has vast off-target effects, low bioavailability and requires high doses to inhibit xCT in vivo and in clinical studies (Timmerman et al., Cancer Cell 2013, 24(4):450-65; Shitara et al., Gastric Cancer 20, 341-349 (2017); Linares et al., Expert Opin Drug Saf. 2011, 10(2):253-63; Robe et al., BMC Cancer 2009, 9:372). Therefore, new therapeutic modalities specifically targeting xCT need to be developed for clinical use.

### SUMMARY

Certain embodiments are directed to therapeutic antibodies, e.g., monoclonal antibodies, that specifically bind xCT epitopes. Certain aspects are directed to monoclonal antibodies (MABs) against xCT peptides and/or epitopes including, but not limited to peptides or epitopes present in extracellular domain (ECD) 1, 2, 3, 4, 6, or various combinations thereof. In certain instances the MABs can be produced using VLP-based vaccination, in particular RNA bacteriophage VLPs. VLPs displaying the various xCT peptides can be used to induce an epitope specific immune response, which can be exploited to produce MABs. In certain aspects, MAB is a IgG2a. kappa or IgG2b, kappa isotype.

In certain aspects the peptide or epitope is or comprises all or a fragment of a peptide having the amino acid sequence MQLIKGQTQNFKDAFSGRDSSITR (SEQ ID NO:81) or KGQTQNFKDAFSGRDSSITRLP (SEQ ID NO:82). Other embodiments are directed to an antibody that specifically binds a peptide having 3, 4, 5, 6, 7, 8, 9, 10, 11, 12, 13, 14, 15, 16, 17, 18, 19, 20, 21, 22, 23, or 24 consecutive amino acids of SEQ ID NO:81 or SEQ ID NO:82. In certain aspects, 1, 2, 3, 4, 5, 6, 7, or more amino acids can be substituted as long as xCT binding specificity of a resulting antibody binding the peptide is maintained. Peptides having amino acid substitutions that do not specifically bind xCT can be specifically excluded. An epitope variant can be a variation on a peptide described herein having 1, 2, 3, 4, 5, 6, 7, or more amino acid substitutions where xCT binding specificity of an antibody produced to the variant peptide is maintained.

The present invention provides high affinity antibodies and antibody fragments that specifically bind xCT, a portion of xCT, an epitope derived from xCT, or an ECD of xCT. As used herein, the term antibody refers to a full length, complete antibody molecule as recognized in the art. The term fragment in the context of the present application refers to a portion of an antibody that retains the capability to bind to xCT, an xCT peptide, or an xCT epitope with high affinity and specificity. Antibody fragments can be defined based on how many domains are included and/or excluded from the original full domain structure. Hence, a fragment can mean Variable heavy (VH) chains, or 1, 2, or 3 VH complementary determining regions (CDRs) or Variable light (VL) or 1, 2, or 3 VL complementary determining regions (CDRs) or Single chain Fv (VH-VL) or Fab (VL-CL-VH-CH1) or Fab2 (VL-CL-VH-CH1)2 or any of the above. In certain aspects, heavy chain CDR1, CDR2, and CDR3 can be defined as 4, 5, 6, 7, 8, 9, 10, 11, 12, 13, 14, 15, or 16 consecutive amino acids of peptide segments define by positions 40 to 54 for CDR1, 65 to 80 for CDR2, and 110 to 125 for CDR 3 of SEQ ID NO:2, 12, 22, 32, 42, 52, 62, 72, 83, 91, or 99. In other aspects, heavy chain CDR1, CDR2, and CDR3 can be consecutive amino acids of peptide segments define by positions 41 to 54 for CDR1, 69 to 78 for CDR2, and 116 to 124 for CDR 3 of SEQ ID NO:2, SEQ ID NO:12, SEQ ID NO:22, SEQ ID NO:32, SEQ ID NO:42, SEQ ID NO:52, SEQ ID NO:62, SEQ ID NO:72, SEQ ID NO:83, SEQ ID NO:91, or SEQ ID NO:99. In certain aspects, light chain CDR1, CDR2, and CDR3 can be defined as 4, 5, 6, 7, 8, 9, 10, 11, 12, 13, 14, 15, or 16 consecutive amino acids of peptide segments define by positions 40 to 65 for CDR1, 70 to 85 for CDR2, and 110 to 125 for CDR 3 of SEQ ID NO:7, SEQ ID NO:17, SEQ ID NO:27, SEQ ID NO:37, SEQ ID NO:47, SEQ ID NO:57, SEQ ID NO:67, SEQ ID NO:77, SEQ ID NO:87, SEQ ID NO:95, or SEQ ID NO:103. In other aspects, heavy chain CDR1, CDR2, and CDR3 can be consecutive amino acids of peptide segments define by positions 43 to 60 for CDR1, 74 to 82 for CDR2, and 113 to 123 for CDR 3 of SEQ ID NO:2, SEQ ID NO:12, SEQ ID NO:22, SEQ ID NO:32, SEQ ID NO:42, SEQ ID NO:52, SEQ ID NO:62, SEQ ID NO:72, SEQ ID NO:83, SEQ ID NO:91, or SEQ ID NO:99. In still further aspects the CDRs of the heavy and light chain antibody portions can be as specifically defined by CDR sequences provided herein.

Any antibody or fragment thereof described herein can be linked or coupled to onr or more agents, such as anticancer moieties (e.g., chemotherapies or radiotherapies), small molecules, PEG, other protein domain(s), or labeling agents. Certain embodiments are directed to an antibody conjugate (AC). An AC can be generally represented by the formula Ab-L-D, wherein Ab is the portion of the conjugate that binds a target (e.g., xCT), L is an optional linker, and D is an agent or a molecule of interest, e.g., a drug portion of the conjugate. The agent or molecule of interest can be a antimitotic cytotoxin, a DNA alkylating agent, a DNA cleaver, a DNA intercalator, a microtubule inhibitor, a topoisomerase inhibitor, chemotherapy, enzyme, radiotherapy, or a detectable label. A preferred example of such a fragment is a single chain antibody variable region fragment (ScFv). The term antibody, as used in this application, generally refers to complete antibody molecules or fragments, unless there is a statement to the contrary. Other xCT binding moieties can be engineered using the amino acid sequence of the complementary determining regions (CDRs) presented on an antibody or non-antibody framework or scaffold.

Antibodies are preferably human, humanized, murine/human chimeric, or ScFv antibodies or fragments thereof. The antibodies and fragments of this invention are further provided as a pharmaceutical preparation for therapeutic use. The invention further provides recombinant DNA molecules encoding xCT antibodies of the invention and expression systems for producing or manufacturing the antibodies recombinantly.

The xCT binding agents (e.g., anti-xCT antibodies) of this invention are useful for treating conditions in which xCT is over-expressed, such as cancer. The antibodies can act through specific and high affinity binding to the cell surface or in vivo expressed xCT. Expression of xCT at the cell membrane is essential for the uptake of cystine required for intracellular glutathione (GSH) synthesis. Therefore, xCT plays an important role in maintaining the intracellular redox balance (Bannai et al., J. Membr. Biol. 89 (1986) 1-8. Patel et al., Neuropharmacology 46 (2004) 273-284.) With the intention of not being bound to any particular theory, the xCT antibodies therapeutic effect may be mediated by inhibiting the uptake of extracellular cystine resulting in decreased intracellular GSH and a corresponding increase of reactive oxygen species. The reduction of GSH also may lead regulated cell death known as ferroptosis. The xCT antibodies can also be used to for cell killing by antibody dependent cellular cytotoxicity (ADCC) and complement dependent cytotoxicity (CDC). Additionally, xCT antibody drug conjugates (ADC) can exert their cell cytotoxicity by binding to cell surface exposed xCT then being internalized and delivering their toxic or therapeutic payload.

Certain embodiments are directed to an xCT antibody that specifically binds an epitope defined by the amino acid sequence of SEQ ID NO:81 and/or SEQ ID NO:82. In certain aspects, an xCT antibody comprises an antibody heavy chain having an amino acid sequence that is or is at least 90, 91, 92, 93, 94, 95, 96, 97, 98, 99, or 100% identical to SEQ ID NO:2, SEQ ID NO:12, SEQ ID NO:22, SEQ ID NO:32, SEQ ID NO:42, SEQ ID NO:52, SEQ ID NO:62, SEQ ID NO:72, SEQ ID NO:83, SEQ ID NO:91, or SEQ ID NO:98. In certain aspects the xCT antibody has a heavy chain comprising a CDR1 having the amino acid sequence of SEQ ID NO:3, SEQ ID NO:13, SEQ ID NO:23, SEQ ID NO:33, SEQ ID NO:43, SEQ ID NO:53, SEQ ID NO:63, SEQ ID NO:73, SEQ ID NO:84, SEQ ID NO:92, or SEQ ID NO:100; a CDR2 having the amino acid sequence of SEQ ID NO:4, SEQ ID NO:14, SEQ ID NO:24, SEQ ID NO:34, SEQ ID NO:44, SEQ ID NO:54, SEQ ID NO:64, SEQ ID NO:74, SEQ ID NO:85, SEQ ID NO:93, or SEQ ID NO:101; and a CDR3 having the amino acid sequence of SEQ ID NO:5, SEQ ID NO:15, SEQ ID NO:25, SEQ ID NO:35, SEQ ID NO:45, SEQ ID NO:55, SEQ ID NO:65, SEQ ID NO:75, SEQ ID NO:86, SEQ ID NO:94, or SEQ ID NO:102. In certain aspects, an xCT antibody comprises an antibody light chain having an amino acid sequence that is or is at least 90, 91, 92, 93, 94, 95, 96, 97, 98, 99, or 100% identical to SEQ ID NO:7, SEQ ID NO:17, SEQ ID NO:27, SEQ ID NO:37, SEQ ID NO:47, SEQ ID NO:57, SEQ ID NO:67, SEQ ID NO:77, SEQ ID NO:87, SEQ ID NO:95, or SEQ ID NO:103. In another aspect the xCT antibody comprises a light chain comprising a CDR1 having the amino acid sequence of SEQ ID NO:8, SEQ ID NO:18, SEQ ID NO:28, SEQ ID NO:38, SEQ ID NO:48, SEQ ID NO:58, SEQ ID NO:68, SEQ ID NO:78, SEQ ID NO:88, SEQ ID NO:96, or SEQ ID NO:104; a CDR2 having the amino acid sequence of SEQ ID NO:9, SEQ ID NO:19, SEQ ID NO:29, SEQ ID NO:39, SEQ ID NO:49, SEQ ID NO:59, SEQ ID NO:69, SEQ ID NO:79, SEQ ID NO:89, SEQ ID NO:97, or SEQ ID NO:105; and a CDR3 having the amino acid sequence of SEQ ID NO:10, SEQ ID NO:20, SEQ ID NO:30, SEQ ID NO:40, SEQ ID NO:50, SEQ ID NO:60, SEQ ID NO:70, SEQ ID NO:80, SEQ ID NO:90, SEQ ID NO:98, or SEQ ID NO:106.

The antibody can comprise a heavy chain comprising a CDR1 having the amino acid sequence of SEQ ID NO:3, a CDR2 having the amino acid sequence of SEQ ID NO:4, and a CDR3 having the amino acid sequence of SEQ ID NO:5; and a light chain comprising a CDR1 having the amino acid sequence of SEQ ID NO:8, a CDR2 having the amino acid sequence of SEQ ID NO:9, and a CDR3 having the amino acid sequence of SEQ ID NO:10.

The antibody can comprise a heavy chain comprising a CDR1 having the amino acid sequence of SEQ ID NO:13, a CDR2 having the amino acid sequence of SEQ ID NO:14, and a CDR3 having the amino acid sequence of SEQ ID NO:15; and a light chain comprising a CDR1 having the amino acid sequence of SEQ ID NO:18, a CDR2 having the amino acid sequence of SEQ ID NO:19, and a CDR3 having the amino acid sequence of SEQ ID NO:20.

The antibody can comprise a heavy chain comprising a CDR1 having the amino acid sequence of SEQ ID NO:23, a CDR2 having the amino acid sequence of SEQ ID NO:24, and a CDR3 having the amino acid sequence of SEQ ID NO:25; and a light chain comprising a CDR1 having the amino acid sequence of SEQ ID NO:28, a CDR2 having the amino acid sequence of SEQ ID NO:29, and a CDR3 having the amino acid sequence of SEQ ID NO:30.

The antibody can comprise a heavy chain comprising a CDR1 having the amino acid sequence of SEQ ID NO:33, a CDR2 having the amino acid sequence of SEQ ID NO:34, and a CDR3 having the amino acid sequence of SEQ ID NO:35; and a light chain comprising a CDR1 having the amino acid sequence of SEQ ID NO:38, a CDR2 having the amino acid sequence of SEQ ID NO:39, and a CDR3 having the amino acid sequence of SEQ ID NO:40.

The antibody can comprise a heavy chain comprising a CDR1 having the amino acid sequence of SEQ ID NO:43, a CDR2 having the amino acid sequence of SEQ ID NO:44, and a CDR3 having the amino acid sequence of SEQ ID NO:45; and a light chain comprising a CDR1 having the amino acid sequence of SEQ ID NO:48, a CDR2 having the amino acid sequence of SEQ ID NO:49, and a CDR3 having the amino acid sequence of SEQ ID NO:50.

The antibody can comprise a heavy chain comprising a CDR1 having the amino acid sequence of SEQ ID NO:53, a CDR2 having the amino acid sequence of SEQ ID NO:54, and a CDR3 having the amino acid sequence of SEQ ID NO:55; and a light chain comprising a CDR1 having the amino acid sequence of SEQ ID NO:58, a CDR2 having the amino acid sequence of SEQ ID NO:59, and a CDR3 having the amino acid sequence of SEQ ID NO:60.

The antibody can comprise a heavy chain comprising a CDR1 having the amino acid sequence of SEQ ID NO:63, a CDR2 having the amino acid sequence of SEQ ID NO:64, and a CDR3 having the amino acid sequence of SEQ ID NO:65; and a light chain comprising a CDR1 having the amino acid sequence of SEQ ID NO:68, a CDR2 having the amino acid sequence of SEQ ID NO:69, and a CDR3 having the amino acid sequence of SEQ ID NO:70.

The antibody can comprise a heavy chain comprising a CDR1 having the amino acid sequence of SEQ ID NO:73, a CDR2 having the amino acid sequence of SEQ ID NO:74, and a CDR3 having the amino acid sequence of SEQ ID NO:75; and a light chain comprising a CDR1 having the amino acid sequence of SEQ ID NO:78, a CDR2 having the amino acid sequence of SEQ ID NO:79, and a CDR3 having the amino acid sequence of SEQ ID NO:80.

The antibody can comprise a heavy chain comprising a CDR1 having the amino acid sequence of SEQ ID NO:84, a CDR2 having the amino acid sequence of SEQ ID NO:85, and a CDR3 having the amino acid sequence of SEQ ID NO:86; and a light chain comprising a CDR1 having the amino acid sequence of SEQ ID NO:88, a CDR2 having the amino acid sequence of SEQ ID NO:89, and a CDR3 having the amino acid sequence of SEQ ID NO:90.

The antibody can comprise a heavy chain comprising a CDR1 having the amino acid sequence of SEQ ID NO:92, a CDR2 having the amino acid sequence of SEQ ID NO:93, and a CDR3 having the amino acid sequence of SEQ ID NO:94; and a light chain comprising a CDR1 having the amino acid sequence of SEQ ID NO:96, a CDR2 having the amino acid sequence of SEQ ID NO:97, and a CDR3 having the amino acid sequence of SEQ ID NO:98.

The antibody can comprise a heavy chain comprising a CDR1 having the amino acid sequence of SEQ ID NO:100, a CDR2 having the amino acid sequence of SEQ ID NO:101, and a CDR3 having the amino acid sequence of SEQ ID NO:102; and a light chain comprising a CDR1 having the amino acid sequence of SEQ ID NO:104, a CDR2 having the amino acid sequence of SEQ ID NO:105, and a CDR3 having the amino acid sequence of SEQ ID NO:106.

In certain aspects, an xCT antibody comprises an antibody heavy chain having an amino acid sequence that is or is at least 90, 91, 92, 93, 94, 95, 96, 97, 98, 99, or 100% identical to SEQ ID NO:2 and an antibody light chain having an amino acid sequence that is or is at least 90, 91, 92, 93, 94, 95, 96, 97, 98, 99, or 100% identical to SEQ ID NO:7. In certain aspects, an xCT antibody comprises an antibody heavy chain having an amino acid sequence that is or is at least 90, 91, 92, 93, 94, 95, 96, 97, 98, 99, or 100% identical to SEQ ID NO:12 and an antibody light chain having an amino acid sequence that is or is at least 90, 91, 92, 93, 94, 95, 96, 97, 98, 99, or 100% identical to SEQ ID NO:17. In certain aspects, an xCT antibody comprises an antibody heavy chain having an amino acid sequence that is or is at least 90, 91, 92, 93, 94, 95, 96, 97, 98, 99, or 100% identical to SEQ ID NO:22 and an antibody light chain having an amino acid sequence that is or is at least 90, 91, 92, 93, 94, 95, 96, 97, 98, 99, or 100% identical to SEQ ID NO:27. In certain aspects, an xCT antibody comprises an antibody heavy chain having an amino acid sequence that is or is at least 90, 91, 92, 93, 94, 95, 96, 97, 98, 99, or 100% identical to SEQ ID NO:32 and an antibody light chain having an amino acid sequence that is or is at least 90, 91, 92, 93, 94, 95, 96, 97, 98, 99, or 100% identical to SEQ ID NO:37. In certain aspects, an xCT antibody comprises an antibody heavy chain having an amino acid sequence that is or is at least 90, 91, 92, 93, 94, 95, 96, 97, 98, 99, or 100% identical to SEQ ID NO:42 and an antibody light chain having an amino acid sequence that is or is at least 90, 91, 92, 93, 94, 95, 96, 97, 98, 99, or 100% identical to SEQ ID NO:47. In certain aspects, an xCT antibody comprises an antibody heavy chain having an amino acid sequence that is or is at least 90, 91, 92, 93, 94, 95, 96, 97, 98, 99, or 100% identical to SEQ ID NO:52 and an antibody light chain having an amino acid sequence that is or is at least 90, 91, 92, 93, 94, 95, 96, 97, 98, 99, or 100% identical to SEQ ID NO:57. In certain aspects, an xCT antibody comprises an antibody heavy chain having an amino acid sequence that is or is at least 90, 91, 92, 93, 94, 95, 96, 97, 98, 99, or 100% identical to SEQ ID NO:62 and an antibody light chain having an amino acid sequence that is or is at least 90, 91, 92, 93, 94, 95, 96, 97, 98, 99, or 100% identical to SEQ ID NO:67. In certain aspects, an xCT antibody comprises an antibody heavy chain having an amino acid sequence that is or is at least 90, 91, 92, 93, 94, 95, 96, 97, 98, 99, or 100% identical to SEQ ID NO:72 and an antibody light chain having an amino acid sequence that is or is at least 90, 91, 92, 93, 94, 95, 96, 97, 98, 99, or 100% identical to SEQ ID NO:77. In certain aspects, an xCT antibody comprises an antibody heavy chain having an amino acid sequence that is or is at least 90, 91, 92, 93, 94, 95, 96, 97, 98, 99, or 100% identical to SEQ ID NO:83 and an antibody light chain having an amino acid sequence that is or is at least 90, 91, 92, 93, 94, 95, 96, 97, 98, 99, or 100% identical to SEQ ID NO:87. In certain aspects, an xCT antibody comprises an antibody heavy chain having an amino acid sequence that is or is at least 90, 91, 92, 93, 94, 95, 96, 97, 98, 99, or 100% identical to SEQ ID NO:91 and an antibody light chain having an amino acid sequence that is or is at least 90, 91, 92, 93, 94, 95, 96, 97, 98, 99, or 100% identical to SEQ ID NO:95. In certain aspects, an xCT antibody comprises an antibody heavy chain having an amino acid sequence that is or is at least 90, 91, 92, 93, 94, 95, 96, 97, 98, 99, or 100% identical to SEQ ID NO:99 and an antibody light chain having an amino acid sequence that is or is at least 90, 91, 92, 93, 94, 95, 96, 97, 98, 99, or 100% identical to SEQ ID NO:103.

In certain aspects the antibody or antibody fragment is human, murine/human chimeric, or humanized. In other aspects the antibody or antibody fragment thereof is a murine/human chimeric antibody. The antibody can be an antibody fragment. In certain aspects the antibody fragment is an ScFv. The ScFv can be murine, human, murine/human chimera, or humanized. The framework of the antibody can be a human antibody frqmeworkd, e.g., a human IgG framework. The antibody can have a framework that is 80, 85, 90, 95, or 98% identical to heavy chain corresponding to GenBank accession number AAA02914.1 (with an amino acid sequence as of the date of earliest priority of this application) and/or light chain corresponding to GenBank accession number ANN81987.1 (with an amino acid sequence as of the date of earliest priority of this application).

This invention provides a therapeutic method or treatment including administering an effective amount of anti-xCT antibodies to a patient that suffers from a disease in which xCT is overexpressed, such as a cancer. Cancers in which xCT is overexpressed include gastrointestinal cancers, including brain, liver, colorectal, pancreatic, stomach and others; lung cancer; breast cancer; leukemias, including acute myeloid leukemias; female reproductive cancers such as cervical, uterine and ovarian cancers; other epithelial cancers such as brain, prostate, liver, and kidney cancers. Particular aspects of the invention are directed to anti-xCT monoclonal antibody that can promote cancer cell killing by inhibiting the uptake of extracellular cystine resulting in decreased intracellular GSH and a corresponding increase of reactive oxygen species. The reduction of GSH can also lead regulated cell death known as ferroptosis. The xCT antibodies can also cell killing by antibody dependent cellular cytotoxicity (ADCC) and complement dependent cytotoxicity (CDC). Additionally, xCT antibody drug conjugates (ADC) can exert their cell cytotoxicity by binding to cell surface exposed xCT then being internalized and delivering their toxic payload to the target tumor cell and bystander tumor cells. In certain aspects, an xCT antibody described herein can be used to detect cancer cells expressing xCT. The cancer cells can be in a biological sample of a subject. In other aspects, a subject can be identified as a subject for treatment using an xCT antibody described herein. In certain aspects, a first xCT antibody can be used as a diagnostic and a therapeutic to treat an identified subject can comprise a second xCT antibody. In certain aspects, the same antibody can be used as a diagnostic and in a therapeutic. The methods described herein can further include contacting a biological sample from a subject comprising cancer cells with a second xCT specific antibody to identify cancer cells expressing xCT prior to administering an anti-xCT antibody, or antibody fragment thereof that binds xCT as a therapy.

The present invention provides for a method of treating cancer comprising administering an effective amount of an anti-xCT antibody or antibody fragment to a patient in need thereof. In certain aspects an xCT antibody drug conjugate is administered to a patient.

In one embodiment, the antibody or antibody fragment is humanized. In a particular embodiment the antibody is a monoclonal antibody or antibody fragment. In another embodiment, the antibody or antibody fragment is chimeric. In another embodiment, the antibody or antibody fragment is an ScFv. In another embodiment, the ScFv is human, murine, or humanized.

In one embodiment, the cancer is at least one selected from the group consisting of gastrointestinal cancer, lung cancer, breast cancer, leukemias, cervical cancer, uterine cancer, ovarian cancers, brain cancer, prostate cancer, liver cancer, and kidney cancer. In another embodiment, the patient is a human or a non-human animal. In another embodiment, the antibody or antibody fragment is administered parenterally, intraperitoneally intravenously or subcutaneous, orally, nasally, via inhalation or rectally. In another embodiment, the antibody or antibody fragment is administered intravenously at a dosage of from 5 mg/m² to 2000 mg/m².

The present invention also provides a method of inducing cell cytotoxicity or ferroptosis in cells expressing xCT comprising contacting the cells with an effective amount of an anti-xCT antibody or antibody fragment or an xCT antibody drug conjugate. In certain aspects the targeted cells are cancer cells.

The present invention also provides a humanized murine anti-xCT antibody or antibody fragment. In a preferred embodiment, the antibody or antibody fragment contains the CDRs of monoclonal antibody that specifically bind the epitopes described herein. In another embodiment, the antibody or antibody fragment is modified with PEG. The present invention also provides an anti-xCT ScFv. In a preferred embodiment, the ScFv contains the CDRs of a monoclonal antibody that specifically bind the epitopes described herein. In another embodiment, the ScFv is modified with PEG. The present invention also provides a fragment of an anti-xCT antibody which has high affinity for xCT. In a preferred embodiment, the fragment contains the CDRs of monoclonal antibody that specifically bind the epitopes described herein. In another embodiment, the fragment is modified with PEG.

The present invention also provides a conjugate in which the antibody or fragment described above is conjugated to at least one other moiety. In certain aspects the moiety is an antimitotic cytotoxin, a DNA alkylating agent, a DNA cleaver, a DNA intercalator, a microtubule inhibitor, a topoisomerase inhibitor, chemotherapy, enzyme, radiotherapy, or a detectable label. In certain aspects the conjugate is an xCT antibody drug conjugate.

The present invention also provides a pharmaceutical composition, comprising the antibody or fragment or conjugate as discussed herein and at least one pharmaceutical excipient. In one embodiment of the invention, the excipient is one or more of water, pH buffers, wetting agents, salts, reducing agents, sugars, glycerol, glycol, oils, preservatives and antimicrobials.

Certain embodiments are directed to a therapeutic antibody that binds an xCT peptide or epitope. In certain aspects VLPs or plasmids are produced that display or encode one or more xCT peptide or epitope, which can be used to induce a therapeutic antibody response in a mammal.

The therapeutic compositions are administered in a manner compatible with the dosage formulation, and in such amount as will be prophylactically and/or therapeutically effective. The quantity to be administered depends on the subject to be treated. Precise amounts of active ingredient required to be administered depend on the judgment of the practitioner. The compositions may be given in a single dose schedule or preferably in a multiple-dose schedule. A multiple-dose schedule is one in which a primary course of administration may be with 1-10 separate doses, followed by other doses given at subsequent time intervals required to maintain and/or reinforce a therapeutic response, for example, at 1-4 months for a second dose and if needed, a subsequent dose(s) after several months.

Administration can be performed, for example, intravenously, orally, nasally, via implant, transmucosally, transdermally, intramuscularly, rectally, and subcutaneously. The following delivery systems, which employ a number of routinely used pharmaceutical carriers, are only representative of the many embodiments envisioned for administering the compositions of the invention. The manner of application may vary. Any of the conventional methods for administration of a polypeptide therapy are applicable. These are believed to include parenterally by injection and the like. The dosage of the composition will depend on the route of administration and will vary according to the size and health of the subject.

The phrases “treating cancer” and “treatment of cancer” mean to decrease, reduce, or inhibit the replication of cancer cells; decrease, reduce or inhibit the spread (formation of metastases) of cancer; decrease tumor size; decrease the number of tumors (i.e., reduce tumor burden); lessen or reduce the number of cancerous cells in the body; prevent recurrence of cancer after surgical removal or other anti-cancer therapies; or ameliorate or alleviate the symptoms of the disease caused by the cancer.

The terms “cancer” and “cancerous”, as used herein, refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The terms “inhibiting,” “reducing,” or “prevention,” or any variation of these terms, when used in the claims or the specification includes any measurable decrease or complete inhibition to achieve a desired result.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. mAb M5 Immunohistochemistry Staining of xCT in Human Tumor Tissues

FIG. 2. xCT mAb M5 Homes and Binds to Tumor Cells in vivo

FIG. 3. xCT Monoclonal Antibodies are Internalized by Human and Murine Tumor Cells

FIG. 4. M5-Tesirine Antibody-Drug Conjugate

FIG. 5. M5-MCVC-PAB-MMAE Antibody-Drug Conjugate

FIG. 6. M5-SMCC-DM1 Antibody-Drug Conjugate

FIG. 7. M5 ADC Cell Cytotoxicity

FIG. 8. M5 ADC Cell Cytotoxicity

FIG. 9. In vivo Activity of M5-Tesirine ADC in a CT26 Xenograft model

FIG. 10. In vivo Activity of M5-Tesirine ADC in a 4T.1 Triple Negative Breast Cancer Xenograft model

FIG. 11. In vivo Activity of M5 ADC in a CT26 Xenograft model

FIG. 12. xCT mAbs Inhibit the Adhesion/Migration of MDA-MB-231 Triple Negative Breast Cancer Cells

FIG. 13. xCT mAbs Inhibit TNBC Spheroid Migration and Invasion.

FIG. 14. Illustrates examples of CDR regions for heavy and light chain portion of representative antibodies.

### DESCRIPTION

Certain cancer cells express abnormally high levels of the plasma cell membrane components of the system x_{c}⁻heterodimeric amino acid transporter specific for cystine/glutamate exchange. System x_{c}⁻ imports L-cystine into the intracellular compartment of a cell, which requires L-cystine for the synthesis of glutathione (L-γ-glutamyl-L-cysteinylglycine, referred to herein as “GSH”), an antioxidant that is important for cell survival under hypoxic conditions, such as those that exist in a tumor environment. The structure of System x_{c}⁻ imports is composed of SLC7A11, a catalytic subunit that gives the transporter its specificity for cystine, and SLC3A2, a regulatory subunit. SLC7A11 and SLC3A2 are also known in the field as xCT and 4F2hc/CD98, respectively.

Because tumor cells, and other abnormally rapidly dividing or differentiating cells require greater amounts of GSH to handle higher levels of oxidative stress, such cells more highly express system x_{c}⁻ components for the importation of cystine than do normal cells under normal conditions. As such, the invention takes advantage of the increased expression of system x_{c}⁻ components by hyperproliferative cells by providing an anti-xCT antibody that targets the xCT component of target cells (e.g., cancer stem cells (CSC).

A non-limiting example of a targeted cancer is triple negative breast cancer (TNBC). TNBC is an aggressive form of breast cancer that lacks the estrogen receptor, progesterone receptor, and HER2 receptor, and accounts for 15-20% of all breast cancers in the US. TNBC has higher rates of relapse and poorer outcomes than other forms of breast cancer and owing to the lack of targetable surface receptors, TNBC are resistant to hormonal and HER2-targeted therapies. The particularly aggressive features of TNBC may be due to the enrichment of cancer stem cells (CSC) that have the unique biological properties necessary for maintenance and spreading of the tumor and through asymmetric division, can differentiate into cancer cells that compose the tumor bulk (Magee et al., Cancer Cell 2012, 21(3):283-96). Due to their resistance to traditional radio- and chemo-therapies (Nagano et al., Oncogene 2013, 32(44):5191-8), CSC represent a reservoir for the relapse, metastatic evolution, and progression of the disease after treatment. Therefore, successful eradication of CSC represents a major barrier towards effective cancer treatments.

### I. THERAPEUTIC ANTIBODIES

Certain embodiments of the present invention is directed to an antibody, e.g., a monoclonal antibody that recognizes human xCT or a cell expressing the same. The invention is also directed to a hybridoma cell line that produces the antibody, and to methods of treating cancer using the antibody. The antibody recognizes and specifically binds human xCT in its native form, which is expressed on the cellular membrane.

The term “antibody” is used herein in the broadest sense and refers generally to a molecule that contains at least one antigen binding site that immunospecifically binds to a particular antigen target of interest. The term “antibody” thus includes but is not limited to antibodies and variants thereof, fragments of antibodies and variants thereof, peptibodies and variants thereof, and antibody mimetics that mimic the structure and/or function of an antibody or a specified fragment or portion thereof, including single chain antibodies and fragments thereof. The term “antibody,” thus includes full-length antibodies or their variants as well as fragments thereof. Binding of an antibody to a target can cause a variety of effects, such as but not limited to, it modulates, decreases, increases, antagonizes, agonizes, mitigates, alleviates, blocks, inhibits, abrogates or interferes with at least one target activity or binding, or with receptor activity or binding, in vitro, in situ, and/or in vivo.

The present invention, thus, encompasses antibodies capable of binding to xCT or portions thereof, including but not limited to Fab, Fab′ and F(ab′)₂, facb, pFc′, Fd, Fv or scFv fragments; diabodies; linear antibodies; single-chain antibody molecules; and multi-specific antibodies formed from antibody fragments.

Accordingly, antibody is used in the broadest sense and specifically covers, for example, single anti-xCT monoclonal antibodies (including agonist, antagonist, and neutralizing antibodies), anti-xCT antibody compositions with polyepitopic specificity, single chain anti-xCT antibodies, and fragments of anti-xCT antibodies.

The term “monoclonal antibody” as used herein refers to an antibody obtained from a population of substantially homogeneous antibodies, i.e., the individual antibodies comprising the population are identical except for possible naturally occurring mutations that may be present in minor amounts. In certain aspects a monoclonal antibody that specifically binds an xCT peptide is described.

Specific antibody fragments of the present invention include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-46) which consists of a single variable, (v) isolated CDR regions, (vi) F(ab′)₂ fragments, a bivalent fragment comprising two linked Fab fragments, (vii) single chain Fv molecules (scFv), wherein a VH domain and a VL domain are linked by a peptide linker which allows the two domains to associate to form an antigen binding site (Bird et al., 1988, Science 242:423-26, Huston et al., 1988, Proc. Natl. Acad. Sci. U.S.A. 85:5879-83), (viii) bispecific single chain Fv (WO 03/11161) and (ix) “diabodies” or “triabodies”, multivalent or multispecific fragments constructed by gene fusion (Tomlinson et. al., 2000, Methods Enzymol. 326:461-79; WO94/13804; Holliger et al., 1993, Proc. Natl. Acad. Sci. U.S.A. 90:6444-48). The antibody fragments may be modified. For example, the molecules may be stabilized by the incorporation of disulfide bridges linking the VH and VL domains (Reiter et al., 1996, Nature Biotech. 14:1239-1245).

“Fv” is the minimum antibody fragment that contains a complete antigen-recognition and binding site. This region consists of a dimer of one heavy-chain and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified to greater than 95% by weight of antibody as determined by the Lowry method, and most preferably more than 99% by weight; to a degree sufficient to obtain at least 15 residues of N-terminal or internal amino acid sequence by use of a spinning cup sequenator; or to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

A “native sequence xCT polypeptide” comprises a polypeptide having the same amino acid sequence as the corresponding xCT polypeptide derived from nature, e.g., SEQ ID NO: 1. Such native sequence xCT polypeptides can be isolated from nature or can be produced by recombinant or synthetic means. The term “native sequence xCT polypeptide” specifically encompasses naturally occurring truncated or secreted forms of the specific xCT polypeptide (e.g., a loop or partial loop sequence), naturally-occurring variant forms (e.g., alternatively spliced forms) and naturally-occurring allelic variants of the polypeptide.

The terms “individual,” “subject,” and “patient,” used interchangeably herein, refer to an animal, preferably a mammalian (including non-primate and primate), including, but not limited to, murines, simians, humans, mammalian farm animals (e.g., bovine, porcine, ovine), mammalian sport animals (e.g., equine), and mammalian pets (e.g., canine and feline); preferably the term refers to humans.

As used herein, the terms “treatment”, “treating”, and the like, refer to obtaining a desired pharmacologic or physiologic effect. The effect may be therapeutic in terms of a partial or complete cure for a disease, symptom, and/or adverse effect attributable to the disease. “Treatment,” as used herein, includes administration of a compound of the present invention for treatment of a disease or condition in a mammal, particularly in a human, and includes: (a) inhibiting the disease, i.e., arresting its development; (b) providing palliative care, i.e., reducing and preventing the suffering of a patient; and (c) relieving the disease, i.e., causing regression of the disease or disorder or alleviating symptoms or complications thereof. Dosage regimens may be adjusted to provide the optimum desired response.

“Framework” or “FR” residues are those variable-domain residues other than the hyper variable region (HVR) residues as herein defined. A “human consensus framework” or “acceptor human framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the VL, the subgroup may be subgroup kappa I, kappa II, kappa III or kappa IV as in Kabat et al, supra. Additionally, for the VH, the subgroup may be subgroup I, subgroup II, or subgroup III as in Kabat et al., supra. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain preexisting amino acid sequence changes. In some embodiments, the number of pre-existing amino acid changes are 10 or less, 9 or less, 8 or less, 7 or less, 6 or less, 5 or less, 4 or less, 3 or less, or 2 or less.

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (K_{D}) of <1×10⁻⁶ M, <1×10⁻⁷ M, <1×10⁻⁸ M, <1×10⁻⁹ M, or <1×10⁻¹⁰ M.

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII Fc expression on hematopoietic cells is summarized in Table 3 on page 464 of Ravetch and Kinet, Annu. Rev. Immunol. 9: 457-92 (1991).

“Binding affinity” generally refers to the strength of the sum total of non-covalent interactions between a single binding site of a molecule (e.g., of an antibody) and its binding partner (e.g., an antigen). Unless indicated otherwise, as used herein, “binding affinity”, “bind to”, “binds to” or “binding to” refers to intrinsic binding affinity that reflects a 1:1 interaction between members of a binding pair (e.g., antibody Fab fragment and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity.

The “K_{D}” or “K_{D} value” according to this invention is in one embodiment measured by surface plasmon resonance using BiaCore® (Cytiva).

The term “conformational epitope” as used herein refers to amino acid residues of the antigen that come together on the surface when the polypeptide chain folds to form the native protein.

A. Monoclonal Antibodies

The anti-xCT antibodies may be monoclonal antibodies. Monoclonal antibodies may be prepared using hybridoma methods, such as those described by Kohler and Milstein (1975) Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

An immunizing agent typically includes the xCT polypeptide, peptide, or a fusion protein thereof. In certain aspects, an immunizing agent is a virus-like particle (VLP) with a xCT peptide displayed on its surface. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding (1986) Monoclonal Antibodies: Principles and Practice, Academic Press, pp. 59-103). Immortalized cell lines may be transformed mammalian cells, particularly myeloma cells of rodent, bovine and human origin. Rat or mouse myeloma cell lines may be employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. (1984) Immunol. 133:3001; Brodeur et al. (1987) Monoclonal Antibody Production Techniques and Applications, Marcel Dekker, Inc., New York, pp. 51-631).

The culture medium in which the hybridoma cells are cultured can then be assayed for the presence of monoclonal antibodies directed against xCT or the xCT peptides described herein. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined using BiaCore®.

The monoclonal antibodies secreted by the subclones may be isolated or purified from the culture medium or ascites fluid by conventional immunoglobulin purification procedures such as protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures, e.g., by using oligonucleotide probes that are capable of binding specifically to genes encoding the heavy and light chains of murine antibodies. The hybridoma cells of the invention serve as a preferred source of such DNA. Once isolated, the DNA may be placed into expression vectors, which are then transfected into host cells, such as, simian COS cells, Chinese hamster ovary (CHO) cells, or myeloma cells that do not otherwise produce immunoglobulin protein, in order to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bifunctional or multifunctional antibody with non-identical antigenic binding specificities, each of which may be monovalent, bivalent, or multivalent.

The antibodies of the present invention may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

The anti-xCT monoclonal antibodies of the invention may be whole or an antigen-binding fragment of the antibody that binds to a xCT polypeptide or peptide, preferably a native sequence xCT polypeptide. Furthermore, in certain embodiments the monoclonal antibody is identified as having recognition of a xCT protein expressed by at least one cancer cell line or tumor tissue.

In one non-limiting embodiment the monoclonal antibody is produced by hybridoma cell line, wherein said antibody or functional fragment thereof binds to a xCT protein and wherein said antibody or functional fragment thereof binds a CSC, neoplastic cell, tumor tissue or antigen thereof as said antibody or functional fragment thereof.

B. Human and Humanized Antibodies

The monoclonal antibodies of the present invention can be human or humanized to reduce the immunogenicity for use in humans. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al. (1986) Nature 321:522; Riechmann et al. (1988) Nature 332:323; and, Presta (1992) Curr. Op. Struct. Biol. 2:593).

Methods for humanizing non-human antibodies are well known in the art. An example approach is to make mouse-human chimeric antibodies having the original variable region of the murine monoclonal antibodies, joined to constant regions of a human immunoglobulin. Chimeric antibodies and methods for their production are known in the art. See, e.g., Cabilly et al., European Patent EP0125023 (published Mar. 3, 2002); Taniguchi et al., European Patent EP0171496 (published May 26, 1993); Morrison et al., European Patent Application EP0173494 (published Jan. 18, 1986); Neuberger et al., International Publication No. WO/1986/01533, (published Mar. 13, 1986); Kudo et al., European Patent Application EP0184187 (published Jun. 11, 1986); Robinson et al., International Publication No. WO/1987/002671 (published May 7, 1987); Liu et al. (1987) Proc. Natl. Acad. Sci. USA 84:3439; Sun et al. (1987) Proc. Natl. Acad. Sci. USA 84:214; Better et al. (1988) Science 240:1041. These references are incorporated herein by reference. Generally, DNA segments encoding the H and L chain antigen-binding regions of the murine mAb can be cloned from the mAb-producing hybridoma cells, which can then be joined to DNA segments encoding C_{H} and C._{L} regions of a human immunoglobulin, respectively, to produce murine-human chimeric immunoglobulin-encoding genes.

A chimeric antibody can be further humanized by replacing sequences of the Fv variable region which are not directly involved in antigen binding with equivalent sequences from human Fv variable regions. General methods for generating humanized antibodies are provided by Morrison, S. L., 1985, Science 229:1202-1207 by Oi et al., 1986, BioTechniques 4:214, and by Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPIIbIIIa antibody producing hybridoma. The recombinant DNA encoding the chimeric antibody can then be cloned into an appropriate expression vector.

Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al. (1986) Nature 321:522; Riechmann et al. (1988) Nature 332:323; Verhoeyen et al. (1988) Science 239:1534), by substituting rodent CDRs or CDR sequences for the corresponding sequences of a human antibody. See also U.S. Pat. No. 5,225,539 and Beidler et al. 1988 J. Immunol. 141:4053. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al. J. Mol. Biol., 222:581 (1991)). The techniques of Cole et al. and Boemer et al. are also available for the preparation of human monoclonal antibodies (Cole et al., Monoclonal Antibodies and Cancer Therapy, Alan R. Liss, p. 77 (1985) and Boerner et al. J. Immunol., 147(1):86 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. Bio/Technology 10:779 (1992); Lonberg et al. Nature 368:856 (1994); Morrison, Nature 368:812 (1994); Fishwild et al. Nature Biotechnology 14:845 (1996); Neuberger, Nature Biotechnology 14:826 (1996); Lonberg and Huszar, Intern. Rev. Immunol. 13:65 (1995).

### II. XCT ANTIBODY CONJUGATES

Certain embodiments relate to the use of an antibody described herein in the preparation of an antibody drug conjugate (ADC). The antibodies described herein can be conjugated to an antimitotic cytotoxin, a DNA alkylating agent, a DNA cleaver, a DNA intercalator, a microtubule inhibitor, a topoisomerase inhibitor, chemotherapy, enzyme, radiotherapy, or a detectable label; and may also be used therapeutically to deliver the toxic or therapeutic agent directly to tumor cells.

An antibody drug conjugate (ADC) is an antibody that is conjugated to a molecule of interest (D) via a linker (L). The antibody-conjugate may be conjugated to one or to more than one molecule of interest (D) via said linker (L). In certain aspects the ADC has the general formula or Ab-L-D, where Ab is an xCT antibody or a binding fragment thereof, L is an optional linker, and D is a molecule of interest.

A molecule of interest can include a reporter molecule, an active substance, an enzyme, a (non-catalytic) protein, a peptide, or an oligonucleotide. An active substance is a pharmacological and/or biological substance, i.e. a substance that is biologically and/or pharmaceutically active, for example a drug or a prodrug, a diagnostic agent, a protein, a peptide, an amino acid, a glycan, a lipid, a vitamin, a steroid, a nucleotide, a nucleoside, a polynucleotide, RNA or DNA. Examples of suitable peptide tags include a cell-penetrating peptides like human lactoferrin or polyarginine.

In certain embodiments, the active substance is selected from the group consisting of drugs and prodrugs. More preferably, the active substance is selected from the group consisting of pharmaceutically active compounds, in particular low to medium molecular weight compounds (e.g. about 200 to about 1500 Da, preferably about 300 to about 1000 Da), such as for example cytotoxins, antiviral agents, antibacterials agents, peptides and oligonucleotides. Examples of cytotoxins include camptothecins, staurosporin, doxorubicin, daunorubicin, colchicine, methotrexate, taxanes, calicheamycins, duocarmycins, indolinobenzodiazepine dimers, maytansines and maytansinoids (i.e. maytansine derivatives), auristatins, tubulysin M, cryptophycin or pyrrolobenzodiazepines (PBDs). Examples of auristatins include dolastatin 10, auristatin F, monomethyl auristatin F (MMAF), auristatin E, monomethyl auristatin E (MMAE), auristatin PE, auristatin TP and auristatin AQ. Examples of maytansines and maytansinoids include mertansine and ansamitocin.

Other useful active substances include chemical compounds useful in the treatment of cancer. Examples of chemotherapeutic agents include SN-38 (7-ethyl-10-hydroxycmptothecin), Erlotinib (TARCEVA®, Genentech/OS I Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNEO, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB 1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin yll and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCINO (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINEO (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

A reporter molecule is a molecule whose presence is readily detected, for example a diagnostic agent, a dye, a fluorophore, a radioactive isotope label, a contrast agent, a magnetic resonance imaging agent or a mass label. Examples of a fluorophore include all kinds of Alexa Fluor® (e.g. Alexa Fluor 488, 555, 673, 680); pH-sensitive pHrodo™ dyes, e.g., green, red; biotin; cyanine dyes (e.g. Cy3 or Cy5); coumarin derivatives; fluorescein; rhodamine; allophycocyanin; and chromomycin.

Example of radioactive isotope label include ⁹⁹mTc, ¹¹¹In, ¹⁸F, ¹⁴C, ⁶⁴Cu, ¹³¹I, or ¹²³I, which may or may not be connected via a chelating moiety such as DTPA, DOTA, NOTA or HYNIC.

In the antibody-conjugate (AC) according to the invention, the molecule of interest can be conjugated to the antibody via a linker (L). Linkers or linking units are well known in the art. “Cross-linker” and “linker” and “cross-linking agent” are used interchangably and in their broadest context to mean a chemical entity used to covalently join two or more entities. For example, a cross-linker joins an antibody to one, two, three, four or more molecule of interest. A linker includes, but is not limited to, the reaction product of small molecule, homo- or hetero-bifunctional, and multifunctional cross-linker compounds, the reaction product of two click-chemistry reactants. It will be understood by one of skill in the art that a cross-linker can refer to the covalently-bound reaction product remaining after the crosslinking of the reactants. The cross-linker can also comprise one or more reactants which have not yet reacted but which are capable to react with another entity. In some embodiments, the linker is a cleavable linker. In some embodiments, the linker is a non-cleavable linker. Useful linkers include, but are not limited to any divalent or multivalent linker known to those of skill in the art. Useful divalent linkers include, but are not limited to alkylene, substituted alkylene, heteroalkylene, substituted heteroalkylene, arylene, substituted arylene, heteroarlyene and substituted heteroarylene. The linker can be a C1-C10 alkylene or C1-10 heteroalkylene. In certain aspects, the linker comprises an amido linker moiety, an amino linker moiety, a carbonyl linker moiety, a carbamate linker moiety, a urea linker moiety, an ether linker moiety, a disulphide linker moiety, a succinamidyl linker moiety, a succinyl linker moiety (e.g., O-succinyl linker moiety), and combinations thereof. In other embodiments, the linker moiety is an ester including: carbonate (—OC(O)O—), succinoyl, phosphate esters (O—(O)POH—O—), sulfonate esters, and combinations thereof. In another embodiment, the linker contains polyethylene glycol (PEG) with an average molecular weight of about 550 to about 10,000 daltons and is optionally substituted by alkyl, alkoxy, acyl or aryl.

Specific representative agents that can be conjugated to an xCT antibody can include, but is not limited to one or more of DM1, a synthetic derivative of the microtubule-targeted agent maytansine (a maytansinoid); SG3199 (SG-3199), a pyrrolobenzodiazepine (PBD); tesirine; DGN549C, a monoimine indolinobenzodiazepine; SN-38, an active metabolite of irinotecan and/or monomethyl auristatin F (MMAF).

Certain embodiments are directed to ADC that include xCT mAb conjugated to Lys-SMCC-DM1; xCT mAb conjugated to SG3199 (SG-3199) dimer warhead component of antibody-drug conjugate (ADC) payload tesirine; xCT mAb conjugated to L-Ala-L-Ala DGN549C, a monoimine indolinobenzodiazepine dimer payload; xCT mAb conjugated to DM21C. DM21, a linker-payload that combines a maytansinoid microtubule disrupting payload with a stable peptide linker; xCT mAb conjugated to a tetra peptide linker with a DXd payload (an exatecan derivative); xCt mAb conjugated to mc-vc-PAB-MMAE Monomethyl auristatin E (MMAE); xCT mAb conjugated to SN-38; and xCT mAb conjugated to monomethyl auristatin F (MMAF).

### III. PHARMACEUTICAL COMPOSITIONS OF ANTIBODIES

In other embodiments there is provided a pharmaceutical composition including an antibody as described above together with a pharmaceutically acceptable carrier, diluent or excipient.

In the preparation of the pharmaceutical compositions comprising the antibodies described in the teachings herein, a variety of vehicles and excipients and routes of administration may be used, as will be apparent to the skilled artisan. Representative formulation technology is taught in, inter alia, Remington: The Science and Practice of Pharmacy, 19th Ed., Mack Publishing Co., Easton, Pa. (1995) and Handbook of Pharmaceutical Excipients, 3rd Ed, Kibbe, A. H. ed., Washington D.C., American Pharmaceutical Association (2000); hereby incorporated by reference in their entirety.

The pharmaceutical compositions will generally comprise a pharmaceutically acceptable carrier and a pharmacologically effective amount of an antibody, or mixture of antibodies.

As used herein, “pharmaceutically acceptable carrier” comprises any standard pharmaceutically accepted carriers known to those of ordinary skill in the art in formulating pharmaceutical compositions. Thus, the antibodies or peptides, by themselves, such as being present as pharmaceutically acceptable salts, or as conjugates, may be prepared as formulations in pharmaceutically acceptable diluents, for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or as solid formulations in appropriate excipients.

The pharmaceutical compositions may further comprise one or more buffers (e.g., neutral buffered saline or phosphate buffered saline), carbohydrates (e.g., glucose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, adjuvants (e.g., aluminium hydroxide), solutes that render the formulation isotonic, hypotonic or weakly hypertonic with the blood of a recipient, suspending agents, thickening agents, and/or preservatives. Alternatively, compositions of the present invention may be formulated as a lyophilisate.

While any suitable carrier known to those of ordinary skill in the art may be employed in the compositions of this invention, the type of carrier will typically vary depending on the mode of administration.

For parenteral administration, the compositions can be administered as injectable dosages of a solution or suspension of the substance in a physiologically acceptable diluent with a pharmaceutical carrier that can be a sterile liquid such as sterile pyrogen free water, oils, saline, glycerol, polyethylene glycol or ethanol. Additionally, auxiliary substances, such as wetting or emulsifying agents, surfactants, pH buffering substances and the like can be present in compositions.

Other components of pharmaceutical compositions are those of petroleum, animal, vegetable, or synthetic origin, for example, non-aqueous solutions of peanut oil, soybean oil, corn oil, cottonseed oil, ethyl oleate, and isopropyl myristate. Antibodies can be administered in the form of a depot injection or implant preparation which can be formulated in such a manner as to permit a sustained release of the active ingredient. An exemplary composition may comprise antibody at 5 mg/ml, formulated in aqueous buffer consisting of 50 mM L-histidine, 150 mM NaCl, adjusted to pH 6.0 with HCl.

Typically, the compositions are prepared as injectables, either as liquid solutions or suspensions, or solid or powder forms suitable for reconstitution with suitable vehicles, including by way of example and not limitation, sterile pyrogen free water, saline, buffered solutions, dextrose solution, etc., prior to injection. The preparation also can be emulsified or encapsulated in liposomes or micro particles such as polylactide, polyglycolide, or copolymers, or other known encapsulating technologies.

The pharmaceutical compositions described herein may be presented in unit-dose or multi-dose containers, such as sealed ampules or vials. Such containers are typically sealed in such a way to preserve the sterility and stability of the formulation until use. In general, formulations may be stored as suspensions, solutions or emulsions in oily or aqueous vehicles, as indicated above. Alternatively, a pharmaceutical composition may be stored in a lyophilized condition requiring only the addition of a sterile liquid carrier immediately prior to use.

A. Uses for Anti-xCT Antibodies

The anti-xCT antibodies of the invention have various utilities. In one embodiment, an anti-xCT is provided for use in a method of treatment of a disease, such as cancer. The method of the invention preferably includes the step of providing an antibody or xCT antigen-binding fragment thereof, as described above, to a subject requiring said treatment.

Methods of immunotargeting cancer cells using antibodies or antibody fragments are well known in the art. U.S. Pat. No. 6,306,393, for instance, describes the use of anti-CD22 antibodies in the immunotherapy of B-cell malignancies, and U.S. Pat. No. 6,329,503 describes immunotargeting of cells that express serpentine transmembrane antigens. Antibodies described herein (including humanized or human monoclonal antibodies or fragments or other modifications thereof, optionally conjugated to cytotoxic or other agents) can be introduced into a patient such that the antibody binds to cancer cells and mediates the destruction of the cells and the tumor and/or inhibits the growth of the cells or the tumor.

Without intending to limit the disclosure, mechanisms by which such antibodies can exert a therapeutic effect may include, for example, complement-mediated cytolysis, antibody-dependent cellular cytotoxicity (ADCC) modulating the physiologic function of the tumor antigen, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, DNA crosslink, DNA alkylation, DNA intercalation, microtubule inhibition, and/or by inducing apoptosis.

In one embodiment, the invention provides a method of treating or preventing a disease comprising administering antibody drug conjugates (ADCs) of the invention to a patient, preferably a human patient. In certain embodiments, the disease to be treated or prevented is a cancer.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

For the prevention or treatment of disease, the appropriate dosage of an ADC will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 0.1 to 20 mg/kg of molecule is an initial candidate dosage for administration to the patient, whether, for example, by one or more separate administrations, or by continuous infusion. A typical daily dosage might range from about 0.1 mg/kg to 20 mg/kg or more, depending on the factors mentioned above. An exemplary dosage of ADC to be administered to a patient is in the range of about 0.1 to about 30 mg/kg of patient weight.

For repeated administrations over several days or longer, depending on the condition, the treatment is sustained until a desired suppression of disease symptoms occurs. An exemplary dosing regimen comprises administering an initial loading dose of about 4 mg/kg, followed by a weekly maintenance dose of about 2 mg/kg of the ADC. Other dosage regimens may be useful. The progress of this therapy is easily monitored by conventional techniques and assays.

An ADC may be combined in a pharmaceutical combination formulation, or dosing regimen as combination therapy, with a second compound having anti-cancer properties. The second compound of the pharmaceutical combination formulation or dosing regimen preferably has complementary activities to the ADC of the combination such that they do not adversely affect each other.

The second compound may be a chemotherapeutic agent, cytotoxic agent, cytokine, growth inhibitory agent, anti-hormonal agent, aromatase inhibitor, protein kinase inhibitor, lipid kinase inhibitor, anti-androgen, antisense oligonucleotide, ribozyme, gene therapy vaccine, anti-angiogenic agent and/or cardioprotectant. Such molecules are suitably present in combination in amounts that are effective for the purpose intended. A pharmaceutical composition containing an ADC may also have a therapeutically effective amount of a chemotherapeutic agent such as a tubulin-forming inhibitor, a topoisomerase inhibitor, or a DNA binder.

Other therapeutic regimens may be combined with the administration of an anticancer agent identified in accordance with this invention. The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein there is a time period while both (or all) active agents simultaneously exert their biological activities.

The combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

Also falling within the scope of this invention are the in vivo metabolic products of the ADC compounds described herein, to the extent such products are novel and unobvious over the prior art. Such products may result for example from the oxidation, reduction, hydrolysis, amidation, esterification, enzymatic cleavage, and the like, of the administered compound. Accordingly, the invention includes novel and unobvious compounds produced by a process comprising contacting a compound of this invention with a mammal for a period of time sufficient to yield a metabolic product thereof.

Metabolites include the products of in vivo cleavage of the ADC where cleavage of any bond occurs that links the drug moiety to the antibody. Metabolic cleavage may thus result in the naked antibody, or an antibody fragment. The antibody metabolite may be linked to a part, or all, of the linker. Metabolic cleavage may also result in the production a drug moiety or part thereof. The drug moiety metabolite may be linked to a part, or all, of the linker.

Treatment is meant to include therapeutic, prophylactic, palliative, or suppressive treatment for the disease, disorder or undesirable condition. Treatment encompasses administration of the subject antibodies in an appropriate form prior to the onset of disease symptoms and/or after clinical manifestations, or other manifestations, of the disease to reduce disease severity, halt disease progression, or eliminate the disease. Prevention of the disease includes prolonging or delaying the onset of symptoms of the disorder or disease, preferably in a subject with increased susceptibility to the disease.

In certain aspects, the therapeutic preparations can use non-modified antibodies or antibodies conjugated with a therapeutic compound, such as a toxin or cytotoxic molecule, depending on the functionality of the antibody. Generally, when non-modified antibodies are used, they will typically have a functional Fc region. By “functional Fc region” herein is meant a minimal sequence for effecting the biological function of Fc, such as binding to Fc receptors, particularly FcγR (e.g., Fcγ RI, FcγRII, and Fcγ RIII).

Without being bound by theory, it is believed that the Fc region may affect the effectiveness of anti-tumor monoclonal antibodies by binding to Fc receptors immune effector cells and modulating cell mediated cytotoxicity, endocytosis, phagocytosis, release of inflammatory cytokines, complement mediate cytotoxicity, and antigen presentation. In this regard, polyclonal antibodies, or mixtures of monoclonals will be advantageous because they will bind to different epitopes and, thus, have a higher density of Fc on the cell surface as compared to when a single monoclonal antibody is used. Of course, to enhance their effectiveness in depleting targeted cells, or where non-modified antibodies are not therapeutically effective, antibodies conjugated to toxins or cytotoxic agents may be used.

The antibody compositions may be used either alone or in combination with other therapeutic agents to increase efficacy of traditional treatments or to target abnormal cells not targeted by the antibodies. The antibodies and antibody compositions of the invention may include, for example, PEGylated antibodies and/or pre-targeting constructs of the antibodies. Combining the antibody therapy method with a chemotherapeutic, radiation or surgical regimen may be preferred in patients that have not received chemotherapeutic treatment, whereas treatment with the antibody therapy may be indicated for patients who have received one or more chemotherapies. Additionally, antibody therapy can also enable the use of reduced dosages of concomitant chemotherapy, particularly in patients that do not tolerate the toxicity of the chemotherapeutic agent very well. Furthermore, treatment of cancer patients with the antibody with tumors resistant to chemotherapeutic agents might induce sensitivity and responsiveness to these agents in combination.

In one aspect, the antibodies are used adjunctively with therapeutic cytotoxic agents, including, by way of example and not limitation, busulfan, thioguanine, idarubicin, cytosine arabinoside, 6-mercaptopurine, doxorubicin, daunorubicin, etoposide, and hydroxyurea. Other agents useful as adjuncts to antibody therapy are compounds directed specifically to the abnormal cellular molecule found in the disease state. These agents will be disease specific.

The amount of the compositions needed for achieving a therapeutic effect will be determined empirically in accordance with conventional procedures for the particular purpose. Generally, for administering the compositions ex vivo or in vivo for therapeutic purposes, the compositions are given at a pharmacologically effective dose. By “pharmacologically effective amount” or “pharmacologically effective dose” is an amount sufficient to produce the desired physiological effect or amount capable of achieving the desired result, particularly for treating or retreating the disorder or disease condition, including reducing or eliminating one or more symptoms or manifestations of the disorder or disease.

As an illustration, administration of antibodies to a patient suffering from cancer provides a therapeutic benefit not only when the underlying disease is eradicated or ameliorated, but also when the patient reports a decrease in the severity or duration of the symptoms associated with the disease. Therapeutic benefit also includes halting or slowing the progression of the underlying disease or disorder, regardless of whether improvement is realized.

The amount administered to the subject will vary depending upon what is being administered, the purpose of the administration, such as prophylaxis or therapy, the state or condition of the subject, the manner of administration, the number of administrations, interval between administrations, and the like. These can be determined empirically by those skilled in the art and may be adjusted for the extent of the therapeutic response. Factors to consider in determining an appropriate dose include, but are not limited to, size and weight of the subject, the age and sex of the subject, the severity of the symptom, the stage of the disease, method of delivery, half-life of the antibodies, and efficacy of the antibodies. Stage of the disease to consider includes whether the disease is acute or chronic, relapsing or remitting phase, and the progressiveness of the disease. Determining the dosages and times of administration for a therapeutically effective amount is well within the skill of the ordinary person in the art.

For any compositions of the present disclosure, the therapeutically effective dose is readily determined by methods well known in the art. For example, an initial effective dose can be estimated from cell culture or other in vitro assays. For example, Sliwkowsky, M X et al. (1999) Semin. Oncol. 26.suppl. 12:60 describes in vitro measurements of antibody dependent cellular cytotoxicity. A dose can then be formulated in animal models to generate a circulating concentration or tissue concentration, including that of the IC50 as determined by the cell culture assays.

In addition, the toxicity and therapeutic efficacy are generally determined by cell culture assays and/or experimental animals, typically by determining the LD50 (lethal dose to 50% of the test population) and ED50 (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the detailed disclosure provided herein. Guidance is also found in standard reference works, for example Fingl and Woodbury, General Principles In: The Pharmaceutical Basis of Therapeutics pp. 1-46 (1975), and the references cited therein.

To achieve an initial tolerizing dose, consideration is given to the possibility that the antibodies may be immunogenic in humans and in non-human primates. The immune response may be biologically significant and may impair the therapeutic efficacy of the antibody even if the antibody is partly or chiefly comprised of human immunoglobulin sequences, for example, in the case of a chimeric or humanized antibody. Within certain embodiments, an initial high dose of antibody is administered such that a degree of immunological tolerance to the therapeutic antibody is established. The tolerizing dose is sufficient to prevent or reduce the induction of an antibody response to repeat administration of the committed progenitor cell specific antibody.

Ranges for the tolerizing dose are, for example, between 10 mg/kg body weight to 50 mg/kg body weight, inclusive. In some embodiments, ranges for the tolerizing dose are between 20 and 40 mg/kg, inclusive. In still other embodiments, ranges for the tolerizing dose are between 20 and 25 mg/kg, inclusive.

Within these therapeutic regimens, the therapeutically effective dose of antibodies may be administered in the range of 0.1 to 10 mg/kg body weight, inclusive. In certain embodiments, therapeutically effective doses are in the range of 0.2 to 5 mg/kg body weight, inclusive. In other embodiments, therapeutically effective doses are in the range of 0.5 to 2 mg/kg, inclusive. Within alternative embodiments, the subsequent therapeutic dose or doses may be in the same or different formulation as the tolerizing dose and/or may be administered by the same or different route as the tolerizing dose.

Antibody compositions may be formulated for any appropriate manner of administration, including for example, oral, nasal, mucosal, intravenous, intraperitoneal, intradermal, subcutaneous, and intramuscular administration.

For the purposes of this invention, the methods of administration are chosen depending on the condition being treated, the form of the subject antibodies, and the pharmaceutical composition.

Administration of the antibody compositions can be done in a variety of ways, including, but not limited to, continuously, subcutaneously, intravenously, orally, topically, transdermal, intraperitoneal, intramuscularly, and intravesically. For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, intraperitoneal administration can be accompanied by intravenous injections. Preferably the therapeutic doses are administered intravenously, intraperitoneally, intramuscularly, or subcutaneously.

The compositions may be administered once or several times. In some embodiments, the compositions may be administered once per day, a few or several times per day, or even multiple times per day, depending upon, among other things, the indication being treated and the judgment of the prescribing physician.

Administration of the compositions may also be achieved through sustained release or long-term delivery methods, which are well known to those skilled in the art. By “sustained release or” “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the antibodies, such as biodegradable polymers described above; pumps, including peristaltic pumps and fluorocarbon propellant pumps; osmotic and mini-osmotic pumps; and the like.

The method of the invention contemplates the administration of single monoclonal antibodies and any antibody that recognizes the particular antigens recognized by these antibodies, as well as combinations, of different monoclonal antibodies. Two or more monoclonal antibodies may provide an improved effect compared to a single antibody. Alternatively, a combination of an antibody with an antibody that binds a different antigen may provide an improved effect compared to a single antibody. Such monoclonal antibodies cocktails may have certain advantages inasmuch as they contain monoclonal antibodies, which exploit different effector mechanisms or combine directly cytotoxic monoclonal antibodies with monoclonal antibodies that rely on immune effector functionality. Such monoclonal antibodies in combination may exhibit synergistic therapeutic effects.

In another embodiment, anti-xCT antibodies may be used in diagnostic assays for xCT, e.g., detecting its expression in specific cells, tissues, organs, or serum.

“Detecting” refers to determining the presence, absence, or amount of an analyte in a sample, and can include quantifying the amount of the analyte in a sample or per cell in a sample.

“Diagnostic” refers to identifying the presence or nature of a pathologic condition. Diagnostic methods differ in their specificity and sensitivity. While a particular diagnostic method may not provide a definitive diagnosis of a condition, it suffices if the method provides a positive indication that aids in diagnosis.

The present invention relates to diagnostic assays, both quantitative and qualitative for detecting levels of xCT polypeptide in cells, organs, tissues and bodily fluids, including determination of normal and abnormal levels. Assay techniques that can be used to determine levels of a polypeptide, such as xCT of the present invention, in a sample derived from a host are well-known to those of skill in the art. Such assay methods include, but are not limited to, radioimmunoassays, immunohistochemistry assays, in situ hybridization assays, competitive-binding assays, Western Blot analyses and ELISA assays. Among these, ELISAs are frequently used to detect a gene's expressed protein in biological fluids. An ELISA assay initially comprises preparing an antibody specific to xCT, preferably a monoclonal antibody. In addition, a reporter antibody generally is prepared which binds specifically to xCT. The reporter antibody is attached to a detectable reagent such as a radioactive, biotin, fluorescent or enzymatic reagent, for example horseradish peroxidase enzyme or alkaline phosphatase.

The above tests can be carried out on samples derived from subjects' bodily fluids and tissue extracts (homogenates or solubilized tissue) such as from tissue biopsy and autopsy material. Levels of xCT, determined in cells and tissues from a patient suspected of suffering from cancer by measuring the polypeptide or by transcription levels, are compared to levels of xCT in normal or control cells or tissues. Increased levels of xCT measured in the subject as compared to levels in the same cells, tissues, or bodily fluids obtained from normal, healthy individuals are indicative of cancer. By “increased levels” it is meant an increase in measured xCT levels in a subject as compared to xCT levels in the same normal cells or tissues. Detection of increased xCT levels is useful in the diagnosis of various cancers including, but not limited to, breast cancer, pancreatic cancer, prostate cancer, melanoma, colon cancer, lung cancer, and thyroid cancer.

Further, monitoring of xCT levels in a subject diagnosed with cancer is useful in determining the onset of metastases in cancers that have not yet metastasized and in determining the stage of the cancer. For example, detection of xCT can be used in a method of monitoring cancer in a subject that has not metastasized for the onset of metastasis. In this method, a subject suffering from a cancer that is not known to have metastasized is identified. xCT levels in a sample from the subject are then measured. These measured xCT levels are then compared with levels of xCT from a normal control sample. An increase in measured xCT levels in the subject versus the normal control is associated with a cancer that has metastasized.

The stage of cancer in a subject suffering from can also be determined. In this method a subject suffering from cancer is identified. xCT levels in a sample of tissue from the patient are measured to establish a baseline xCT level for said patient. xCT levels in samples of the same tissue are then determined at subsequent time periods such as scheduled check-ups with the subject's physician. Measured xCT levels are then compared with the baseline xCT levels for the patient. In this method, an increase in measured xCT levels in the subject versus baseline xCT levels in the subject is associated with a cancer that is progressing and a decrease in measured xCT levels versus baseline xCT levels is associated with a cancer that is regressing or in remission. Increases in measured xCT levels as compared to baseline xCT levels established for the subject may also be indicative of metastases.

In one embodiment, xCT immunohistochemistry functions as an “index diagnostic” to assign risk based on the presence of xCT expression. Therefore, based on this and other parameters (e.g., size of lesion), one can determine whether or not different therapeutic modalities (i.e., chemotherapy, radiation therapy, surgery) should be used. In a related aspect, methods for monitoring progression of pre-malignancy into a malignant phenotype are disclosed. For example, by using serial sampling (i.e., biopsy) of the tissue and observing the state of xCT expression in the lesions, one can determine whether or not the pre-malignancies are progressing in a way that would indicate whether therapeutic intervention is advised or is successful.

One aspect of the invention is a method to determine the likelihood of a group of cells to become cancerous, e.g., the cells or glands become pre-malignancies or progress to cancerous lesions. The invention utilizes an agent, such as an antibody, that specifically binds to xCT protein to assess levels of xCT in tissue and cells. xCT expression in cells and tissue may also be assessed using nucleic acid analysis, such as selective amplification, or hybridization methods, immunohistochemistry, and western blot. A level of xCT above normal or control levels, indicates an increased likelihood that premalignant disease is present, i.e., that the cells or tissues are premalignant.

B. Antibody Kits

Antibody kits are provided which contain the necessary reagents to carry out the treatments or assays of the present invention. The kit may include one or more compartments, each to receive one or more containers such as: (a) a first container comprising one of the components of the present invention described above; and (b) one or more other containers comprising one or more of the following: wash reagents, reagents capable of detecting presence of the antibody or peptide.

The containers allow the efficient transfer of reagents from one compartment to another compartment such that the samples and reagents are not cross-contaminated, and the agents or solutions of each container can be added in a quantitative fashion from one compartment to another.

The kit typically contains containers that may be formed from a variety of materials, such as glass or plastic, and can include for example, bottles, vials, syringes, and test tubes. A label typically accompanies the kit, and includes any writing or recorded material, which may be in electronic or computer readable form (e.g., disk, optical disc, or tape) providing instructions or other information for used of the contents of the kit. The label indicates that the formulation is used for diagnosing or treating the disorder of choice.

One skilled in the art will readily recognize that the disclosed antibodies of the present invention can be readily incorporated into one of the established kit formats that are well known in the art.

### IV. ANTI-CANCER THERAPIES

In certain embodiments the compositions and methods described herein in can be administered in conjunction or combination with other anti-cancer therapies for the treatment of cancer. Therapeutically effective doses can be determined by one of skill in the art and will depend on the severity and course of the disease, the patient's health and response to treatment, the patient's age, weight, height, sex, previous medical history and the judgment of the treating physician.

In some methods of the invention, the cancer cell is a tumor cell. The cancer cell may be in a patient. The patient may have a solid tumor. In such cases, embodiments may further involve performing surgery on the patient, such as by resecting all or part of the tumor. xCT VLPs described herein can be administered before, during, or after an anti-cancer treatment. Anti-cancer treatments may be administered to the patient before, after, or at the same time as surgery. In additional embodiments, patients may also be administered directly, endoscopically, intratracheally, intratumorally, intravenously, intralesionally, intramuscularly, intraperitoneally, regionally, percutaneously, topically, intrarterially, intravesically, or subcutaneously. Anti-cancer compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

Methods of treating cancer may further include administering to the patient chemotherapy or radiotherapy, which may be administered more than one time.

Chemotherapy includes, but is not limited to, docetaxel, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, methotrexate, gemcitabine, oxaliplatin, irinotecan, topotecan, or any analog or derivative variant thereof. Radiation therapy includes, but is not limited to, X-ray irradiation, UV-irradiation, γ-irradiation, electron-beam radiation, or microwaves. Moreover, a cell or a patient may be administered a microtubule stabilizing agent, including, but not limited to, taxane, as part of methods of the invention. It is specifically contemplated that any of the compounds or derivatives or analogs, can be used with these combination therapies.

In some embodiments, the cancer that is administered the composition(s) described herein may be a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterus cell. In certain aspects the cancer is breast cancer.

### V. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Monoclonal Antibody Production and Selection

The sequence of the third domain of human xCT protein SEQ ID NO:81 or SEQ ID NO:82 was expressed within the coat protein AB-loop of MS2 to produce the AX09 VLP. Briefly, the ECD3 sequence was cloned in the pDSP62 plasmid which was electroporated into the E. coli C41 DE3 or BL21AI cells, grown to mid-log phase and VLP expression was induced by the addition of IPTG (1 mM, Sigma-Aldrich, St. Louis, Mo., USA) for 3 hours at 28° C. Bacteria pellets were lysed in lysis buffer (50 mM Tris-HCl, pH 8.5, 100 mM NaCl, 10 mM EDTA, Sigma-Aldrich), sonicated and purified from bacterial debris by centrifugation. Bacterial DNA was removed by treating the supernatant with DNase I (10 units/mL, 1 hour at 37° C.; Sigma-Aldrich) and the VLPs were purified by size exclusion chromatography (Sepharose CL-4B resin, Sigma-Aldrich). Buffer exchange (Phosphate-buffered saline, PBS) and concentration of purified VLPs were achieved using ultrafiltration (Amicon Ultrafiltration device, 100 Kd MWCO, Sigma-Aldrich) and resulting VLP preparations were quantified by Bradford assay (BioRad Philadelphia, Pa., USA). VLP purity was assessed by agarose and SDS-PAGE gel electrophoresis. Purified AX09 VLP (10 μg) was used to immunize Balb/c mice. Following the primary immunization the mice were boosted twice and four days post the last boost the spleens from the immunized mice were harvested use to prepare splenocytes. Logarithmic growth phase SP2/0 cells were collected in 50 ml centrifuge tube and centrifuged it (5 min, 200×g). The SP2/0 myeloma cell were mixed with the splenocytes at a ratio of 1:3. The mixture was then electroporated and after the fusion the cells were placed in HAT medium, hypoxanthine, thymidine, aminopterin, FBS and DMEM pH 7.0. 100 μl/well of cell suspension was added to each well of a 96-well fusion plate coated with intraperitoneal feeder cells and incubated at 37° C., 6.5% CO₂ in humidified incubator.

Hybridoma supernatants were screened by ELISA for the production of antibodies that recognize SEQ ID NO 81 or SEQ ID 82. Briefly, the ELISA assay included adding 250 ng of AX09 VLP or MS2 VLP in a total of 100 μL to Immulon II 2HB 96-well ELISA plates and incubated overnight at 4° C. The following morning, plates were washed with 1×PBS and blocked with 0.5%, milk in 1×PBS for 1 hr with rocking at RT. Plates were washed and incubated with the diluted clone supernatants (2-fold dilutions starting at 5 ng) or control sera (2-fold dilutions starting at 1:5000) for 2 hr with rocking at RT. After washing, HRP-labeled goat anti-mouse IgG (1:5000) was added to wells and incubated at RT for 1 hr with rocking. Wells were washed and 100 μL of TMB Soluble (Calbiochem) were added and incubated at RT for 10 min with rocking. The reaction was stopped with the addition of 50 μL 2% HCL and the plates were read at 450 nm.

Each positive hybridoma that was producing anti-AX09 antibodies was cloned and subcloned using the limiting dilution technique. Based on these results the multiple subclones were selected and proceeded to clone stabilization, antibody production, and sequencing: 7C1E1H5 (M1), 17H12EG6 (M5), 18C1H18F4D7 (M7), 10A12C11H2E8 (M8), 21B8B2H6H11 (M9), 19F4H10E7 (M30), 27D10G2G2 (M11), 41H7C11C10 (M40), H1E1, H1B9, H1D5, H1A5, H1A1.

### Example 2

### Scale Up of xCT Monoclonal Antibodies

Transient transfection for expression of clone 17H12EG6 (M5) was conducted using Expi293F™ cells (ThermoFisher). Briefly, Expi293F cells were grown in Epi203 expression media (Gibco) and the heavy chain expression plasmid (17H12Eg6-HC-mIgG2a-pcDNA 3.4) and the light chain expression plasmid (17H12Eg6-LC-mIgG2a-pcDNA 3.4) plus Expi293 transfection reagent (Gibco) were mixed for 20 min at room temperarure and then the transvection mixture was added to the 3.0×10⁶ Expi293F™ cells/ml and incubated for 17 hr at 37° C., 8% CO₂. After 6 days the supernatant was collected by centrifugation and the IgG purified by Monofinity A resin (Genscript).

### Example 3

### mAb M5 Immunohistochemistry Staining of xCT in Human Tumor Tissues

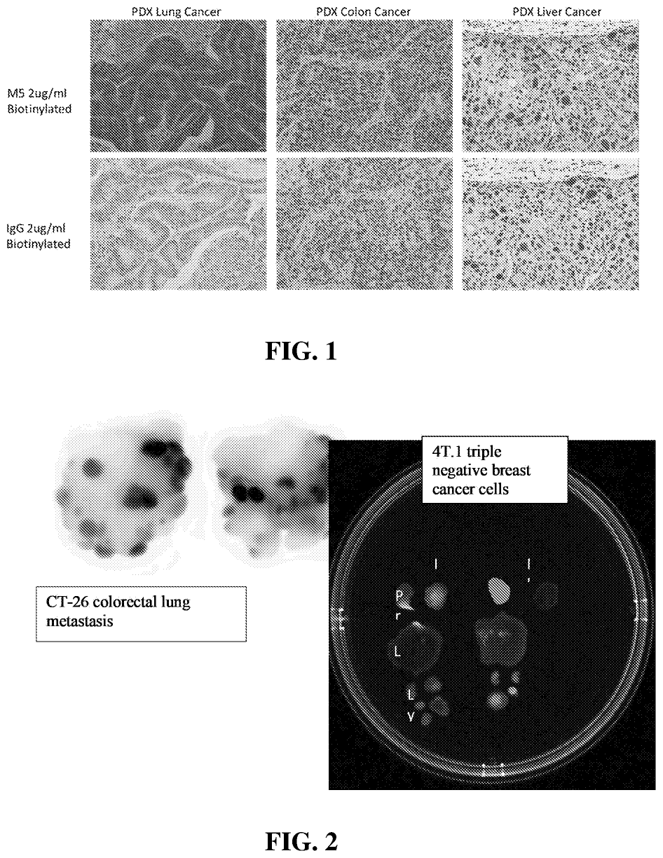

The ability of M5 to specifically recognize xCT in a variety of samples from both normal and cancer (tumor) formalin fixed paraffin embedded tissues was evaluated through the use of immunohistochemistry (IHC). Lung, liver and colorectal tumor samples were obtained and the expression of xCT was determined using the xCT mononclonal antibody M5. An IgG₂ₐ isotype was used as negative control. Briefly, slides were incubated at 60° C. for 30 min on a hot plate in order to melt the paraffin present in the sample. A hydration process that consist in 2 changes of 100% xylene, followed by consecutive washes (5 minute each) of 100% ethanol, 90% ethanol, 80% ethanol, 70% ethanol, 50% ethanol and distillated water, was performed avoiding excessive shaking of the slides. A subsequent epitope retrieval was performed using a boiling Target Retrieval Solution (Agilent-Dako Cat #S1700) for 30 min and cooled down at room temperature for 20 min and the excess of retrieval buffer was removed from each slide. All the following steps, with an exception of incubation of primary antibody (4° C.) were done at room temperature. After washing 3 times with 1×PBS, the tissue was permeabilized using 0.4% Triton diluted in 1×PBS for 3 min and rinsed with 1×PBS 3 times for 5 min each. Unspecific binding was blocked with 5% BSA in 1×PBS for 1 hour. The sections then were incubated with biotinylated M5 IgG or control IgG at a concentration of 2 μg/ml diluted in blocking buffer overnight at 4° C. in a humidified chamber. Once sections were washed 3 times with 1×PBS, an incubation for 10 min with a solution of H₂O₂ at 0.3% was used to block endogenous peroxidase activity. Subsequently, after the H₂O₂ was removed and washed, all sections were incubated for 45 min to 1 hour with streptavidin-HRP conjugated secondary antibody diluted in blocking buffer. Then the sections were extensively washed at least for 10 min and staining was visualized as brown staining by using 3,3-Diaminobenzidine (Vector Cat #SK4100). Then, the slides were washed and nuclear counterstained with hematoxylin dilution in water (1:3) for no more than 30 seconds/slide. Lastly, a dehydration process was performed starting with distillated water going through washes with ethanol dilution of 50%, 70%, 90%, 100% and cleared with xylene 100%. Every wash was done for 3 min. Slides were partially dryed and mounted using Thermo Scientific Richard-Allan Scientific Mounting Media (Cat #4112). Once the mounting media was totally dry photographs were taken using a light microscope (Leica DM 750). xCT staining was detected in tumor sections both subcutaneous as well as PDX samples (medium to intense brown staining) whereas in normal (FIG. 1) tissue as well as tumor adjacent tissue little to no staining was observed. In addition, no staining was detected in any of the samples analyzed when the IgG2a isotope control was used under the same conditions used for M5. The data clearly indicate significant differences in the expression of xCT between normal tissue and tumor samples and that overexpression of xCT may be a good biomarker to identify cancer patients that would benefit from xCT antibody treatment.

### Example 4

### xCT Monoclonal Antibodies Bind to Tumor Cells In Vivo

xCT monoclonal antibody M5 in vivo binding to 4T.1 mammary carcinoma cells and CT-26 undifferentiated colon carcinoma cells was demonstrated using a mouse model. When tumors reached 10 mm×9 mm antibodies (20 μg of Alex Fluor-660® labeled M5 or IgG-control) were injected IV. After 24 hrs the mice were perfused with PBS. The primary 4T.1 tumor, lymph nodes, and lungs and the lungs from the CT-26 challenged mice were explanted and analyzed ex vivo using the GE/Amersham Imager 600RGB. As can be seen in FIG. 2. The labeled M5 was able to home in vivo to the 4T.1 primary tumor, tumors in the lymph nodes and lung metastasis. Similarly, the labeled M5 was able to detect CT-26 lung metastasis. These data suggest that xCT antibodies could be used as a diagnostic to detect primary tumors and track the metastatic spread and invasion of distant tissues/organs. These data also suggest that xCT antibody drug conjugates delivered intravenously could be used to treat both the primary tumor as well as tumor cells that have spread from the original site.

### Example 5

### xCT Antibodies Internalize in xCT Expressing Tumor Cell Lines

Receptor-mediated internalization is a critical characteristic of therapeutic ADCs. Upon binding membrane target, the antibody-receptor complex can initiatively translocate into the cytosol via endocytosis, resulting in either degradation or recycling. In addition, antibodies with internalization functions can act as excellent vehicles for targeted delivery of drugs, toxins, into cells for many therapeutic/diagnostic applications.

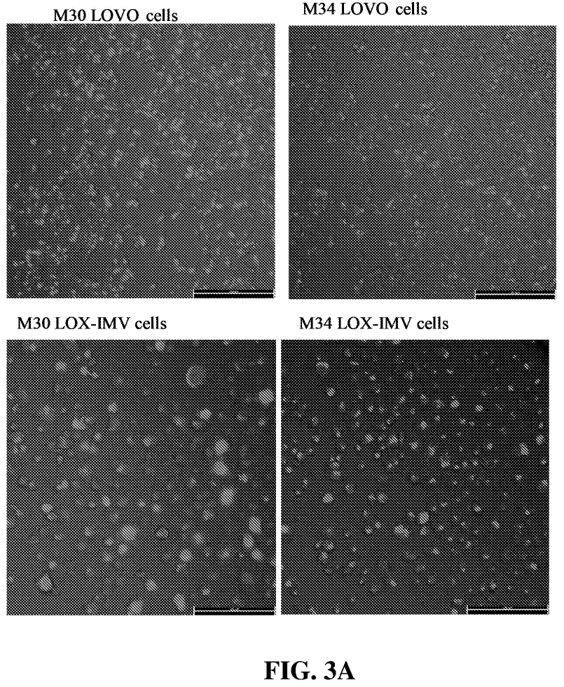

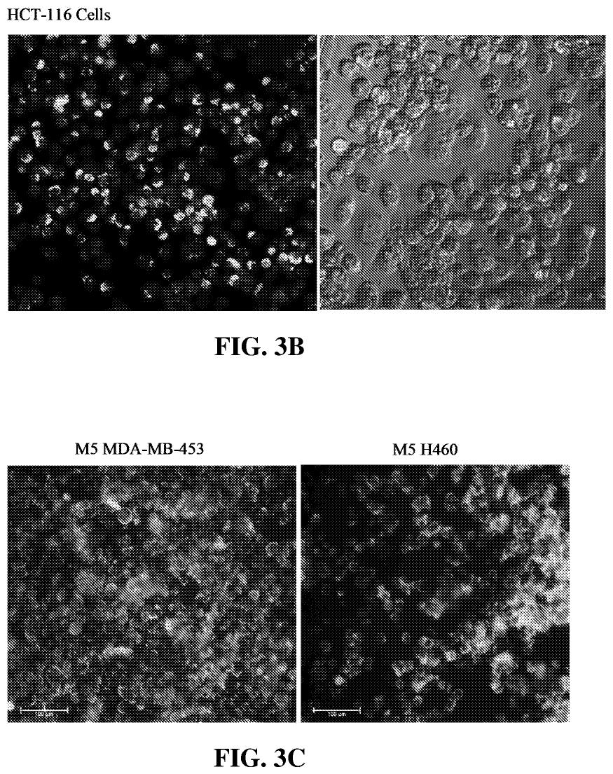

To determine the internalization of the xCT monoclonal antibodies, purified IgG was labeled by using the pH-sensitive pHrodo iFL green or red STP ester dye according to the manufacturer's instructions (ThermoFisher). pHrodo iFL dyes dramatically increase their fluorescence as the pH of its surroundings become more acidic. Briefly, 100 ug of each antibody (in 0.1 M sodium bicarbonate buffer, pH 8.4) were reacted with the solution of amine-reactive pHrodo™ iFL dye (resuspended in anhydrous DMSO), adding the appropriate amount of pHrodo™ iFL dye to the IgG in 5 molar excess of pHrodo™ iFL dye and allowed to react for 60 minutes at room temperature in the dark. Unreacted dye was removed from the conjugate by using size exclusion purification. The internalization assay was performed as following; HCT-116 cells (human colorectal carcinoma), LOVO cells (human metastatic colorectal), LOX-IMV cells (human melanoma), MDA-MB-453 cells (triple negative breast cancer cells, H460 cells (large cell lung carcinoma) (were plated in culture media (supplemented with 5% FBS ultra-low IgG and 1 μg of Hoechst 33342) at the density of 2500 cells per well of tissue culture-treated 96-well plates, 10 μg/mL of the labeled antibodies were added to the cells and allowed to adhere and growth for 36-48 hours at 37° C. Cells were imaged using fluorescent microscopy (blue Hoechst dye for nuclei, red or green fluorescence internalized xCT antibodies). As demonstrated in FIG. 3, monoclonal antibodies M5, M30, and M34 were efficiently internalized indicating that they would be excellent candidate for the production of an ADC.

### Example 6

### Binding Affinity of xCT Monoclonal Antibodies

Surface plasmon resonance (BiaCore®) was used to determine the binding kinetics of xCT monoclonal antibodies to the human ECD3 peptide (Seq ID NO 51). Briefly, biotinylated ECD3 peptide (100 ng/ml) was captured by high-affinity streptavidin (SA) sensor chip (Cytiva). The unbound peptide was washed from the sensor chip with PBS. Once a baseline had been established, the monoclonal antibodies (23 ng/ml) were injected and allowed to bind the immobilized peptide. The resulting sensorgrams were analyzed and the data are listed. As shown in Table 1, high affinity binding of the monoclonal antibodies to the xCT ECD3 peptide was detected.

TABLE 1
High affinity binding of the monoclonal
antibodies to the xCT ECD3 peptide

| | 1/Ms | 1/s | |
|---|---|---|---|
| | Ka | kd | KD |
| M5 | 1.55E+08 | 0.004004 | 2.59E−11 |
| M32 | 5.49E+08 | 0.1675 | 3.05E−10 |
| M30 | 3.97E+09 | 138.8 | 3.50E−08 |
| M16 | 2.38E+10 | 89.11 | 3.74E−09 |
| M6 | 2.06E+08 | 2.112 | 1.03E−08 |
| M43 | 3.61E+07 | 4.61E−04 | 1.27E−11 |
| M15 | 7.96E+06 | 0.106 | 1.33E−08 |
| M9 | 1.08E+07 | 7.75E−05 | 7.17E−12 |

### Example 7

### xCT Antibody Drug Conjugates (ADC)

Certain embodiments of this invention is an xCT antibody-linker-drug or ADC in which the drug or molecule of interest belongs to the following classes: camptothecins, indolinobenzodiazepine dimers, maytansines and maytansinoids, auristatins, or pyrrolobenzodiazepine and the antibody is 7C1E1H5 (M1), 17H12EG6 (M5), 18C1H18F4D7 (M7), 10A12C11H2E8 (M8), 21B8B2H6H11 (M9), 27D10G2G2 (M11), 19F4H10E7 (M30), 41H7C11C10 (M40).

Conjugation of Hybridoma-Tesirine Antibody-Drug Conjugate.

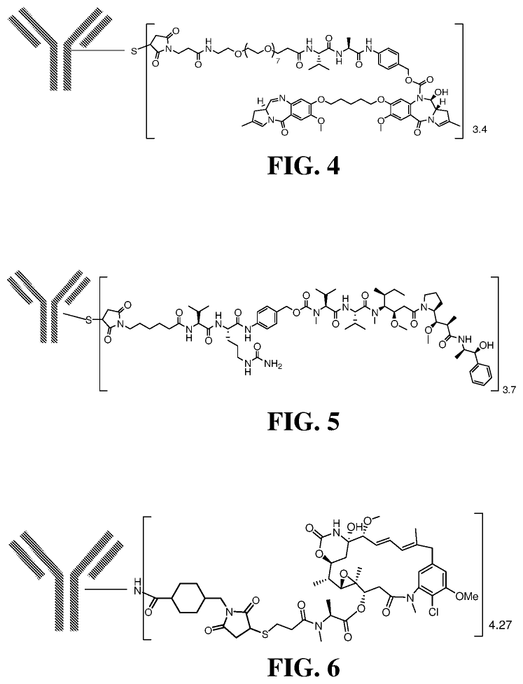

For each of these batches two 2 mL vials of 4.6 mg/mL M5 IgG2a were thawed from the −80° C. freezer to room temperature. Zeba desalting columns (7 kDa MWCO) were washed/equilibrated with conjugation buffer (1× Phosphate buffered saline (PBS) pH 7.5, 1 mM Diethylenetriamine pentaacetate (DTPA) 10% v/v Dimethyl acetamide (DMA) by centrifuging them 3 times at 2000×g for 3 minutes each. The antibody was buffer exchanged through centrifugal gel filtration into conjugation buffer. After buffer exchange, the concentration was confirmed with the Nanodrop and 1.407 extinction coefficient. The antibody solution was diluted down to 3 mg/mL and chilled to 4° C. in ice. To reduce the antibody, 7× molar excess 100 mM TCEP was added and the antibody solution mixed on a rotator for 2 hours in the fridge at 4° C. After reduction, Zeba columns that were washed in the same manner as earlier were used to remove TCEP from the antibody solution. Another 10% DMA was added to the antibody solution to bring the total percent of DMA in solution up to 20%. To conjugate the antibody, 5 equivalents, based on moles of antibody, of 10 mM tesirine was added to the reaction mixture and conjugated for 2 hours at room temperature while mixing on a rotator. Zeba columns were washed with centrifuge with 1×PBS buffer 3 times (2000×g for 3 minutes). The antibody solution was loaded onto the washed Zeba columns and the payload was removed by centrifuging at 2000×g for 3 minutes. ADCs were stored at 4° C. after conjugation was completed. The conjugates were analyzed with RPLC-MS and individual batches of ADCs were purified by SEC to remove the aggregates and excess payload-linker. The conjugation was carried out twice to prepare the required amount of the conjugate and the two batches of purified ADCs solutions were pooled and concentrated with Vivaspin20 10 kDa MWCO centrifugal concentrators. The pooled material was analyzed again with RPLC-MS, filtered, and ran on a SEC column for a final profile. Finally, a Charles River Endochrome-K LAL assay kit and a 96-well plate was used to test for endotoxin in the sample (FIG. 4).

Conjugation of Hybridoma-MCVC-PAB-MMAE Antibody-Drug Conjugate.

Three, 2 mL vials of 4.6 mg/mL M5 IgG2a were thawed from −80° C. freezer to room temperature. Zeba desalting columns were washed with conjugation buffer (1×PBS pH 7.5 1 mM DTPA 10% v/v DMA) by centrifuging them 3 times at 2000×g for 3 minutes. The antibody was buffer exchanged through centrifugal gel filtration into conjugation buffer. After buffer exchange the concentration was confirmed with the Nanodrop and 1.407 extinction coefficient. The antibody solution was diluted down to 3 mg/mL and chilled to 4° C. in ice. To reduce the antibody, 7× molar excess 100 mM TCEP was added and the antibody mixed on a rotator for 2 hours in the fridge at 4° C. After reduction, Zeba columns that were washed in the same manner as earlier were used to remove TCEP from the antibody solution. To conjugate the antibody, 7 equivalences, based on moles of antibody, of 10 mM MCVC-PAB-MMAE was added to the reaction mixture and left to conjugate for 2 hours at room temperature while mixing on a rotator. Zeba columns were washed with centrifuge with 1× PBS buffer 3 times (2000×g for 3 minutes). The antibody solution was loaded onto the washed Zeba columns and the payload was removed by centrifuging at 2000×g for 3 minutes. ADCs were stored at 4° C. after conjugation was completed. The conjugates were analyzed with HIC and RPLC-MS. A SEC column was used to purify and remove the aggregates. Conjugates were concentrated down with Vivaspin20 10 kDa MWCO centrifugal concentrators after purification and analyzed on HIC and RPLC-MS again. The conjugate was filtered and ran on SEC column for final profile. Finally, a Charles River Endochrome-K LAL assay kit and a 96-well plate was used to test for endotoxin in the sample (FIG. 5).

Conjugation of Hybridoma-SMCC-DM1 Antibody-Drug Conjugate.

For each of these batches two 2-mL vials of 4.6 mg/mL M5 IgG were thawed from the −80° C. freezer to room temperature. Zeba desalting columns (7 kDa MWCO) were then washed/equilibrated with conjugation buffer (lx Phosphate buffered saline (PBS) pH 7.5, 1 mM Diethylenetriamine pentaacetate (DTPA) 10% v/v Dimethyl acetamide (DMA)) by centrifuging them 3 times at 2000×g for 3 minutes each. The antibody was then buffer exchanged through centrifugal gel filtration into conjugation buffer. After buffer exchange, the concentration was confirmed with the Nanodrop and 1.407 extinction coefficient. To conjugate the antibody, 10 and 12 equivalents, based on moles of antibody, of 10 mM SMCC-DM1 was added to the reaction mixture and conjugated for 2 hours at room temperature while mixing on a rotator. After conjugation, samples pipetted into Amicon 10 kDa centrifugal devices and diluted 15-fold with PBS. Samples centrifuged at 4,000×g for 30 min. Samples concentrated back to original volume and diluted again with PBS, then concentrated a second time back to original volume via centrifugal devices. 100 μg of each test conjugate digested overnight with PNGaseF at 37° C. Average DAR for each conjugate calculated via SEC-MS. Test conjugates pooled and concentrated then purified over SEC column, monomer fractions pooled and stored at 4° C. (FIG. 6).

### Example 8

### M5-Tesirine Cell Cytotoxicity Assay

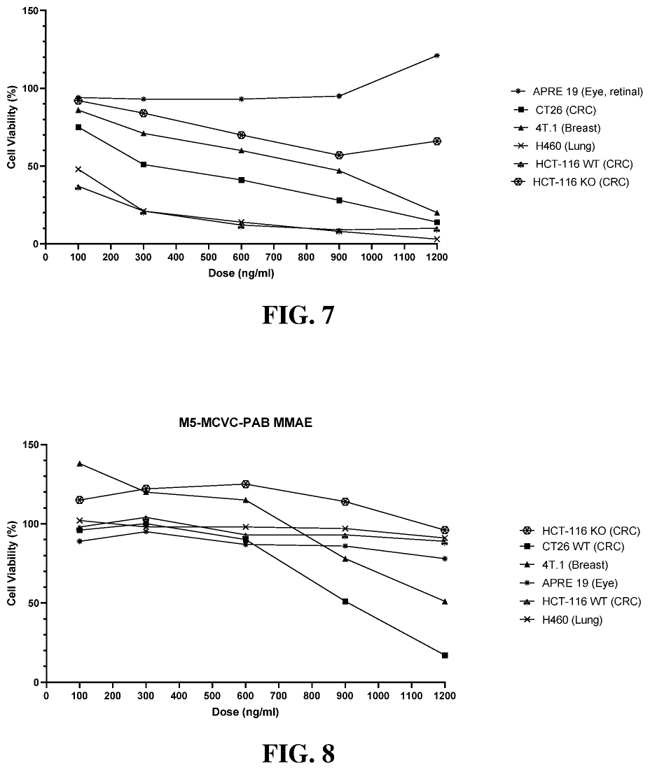

To determine the ability of M5-tesirine and M5-MCVC-PAB MMAE to kill cancer cells in vivo, xCT-positive and xCT-negative cancer cell lines were plated in culture media (supplemented with 3% FBS ultra-low IgG) at a density of 2,000-4,000 cells per well of tissue culture-treated 96-well plates and allowed to adhere overnight at 37° C./5% CO₂. M5-tesirine and M5-MCVC-PAB MMAE was serially diluted and added to the wells and incubated at 37° C./5% CO₂ for 120 hours. Cell Titer-Glo® reagent (Promega, Madison, Wis.) was added to each well and incubated for 10 minutes at room temperature with mild shaking. The luminescence of each sample was read at 560 nm using a Cytation 5 (BioTek). As shown in FIG. 7, M5-tesirine exhibited potent cytotoxicity against multiple cancer cell lines. M5-MCVC-PAB MMAE showed lower potency for most cancer cell lines at the doses tested. Importantly, there was little to no cytotoxicity observed for the xCT negative cell line APRES 19 retina cell line or HCT-116 knock-out. Each cell line was tested in triplicate at each dilution.

### Example 9

### In Vivo Efficacy of M5-Tesirine ADC in a CT-26 Xenograft Model

All animal work was performed in an AAALAC accredited facility, following a protocol approved by the facility's IACUC committee. On Day 0, female Balb/c mice (seven weeks old, Charles River) were implanted subcutaneously into the 4^{th} mammary fat pad on the left side, using 500,000 CT-26 cells. Animals were monitored and when average tumor volume reached 280-310 mm³ on Day 15, mice were randomized into treatment groups and dosed in a 100 μL volume with the following:

Group A—3.0 mg/kg M5-tesirine ADC
Group B—1.0 mg/kg M5-tesirine ADC
Group C—0.1 mg/kg M5-tesirine ADC
Group D—unconjugated M5

### Group E—PBS

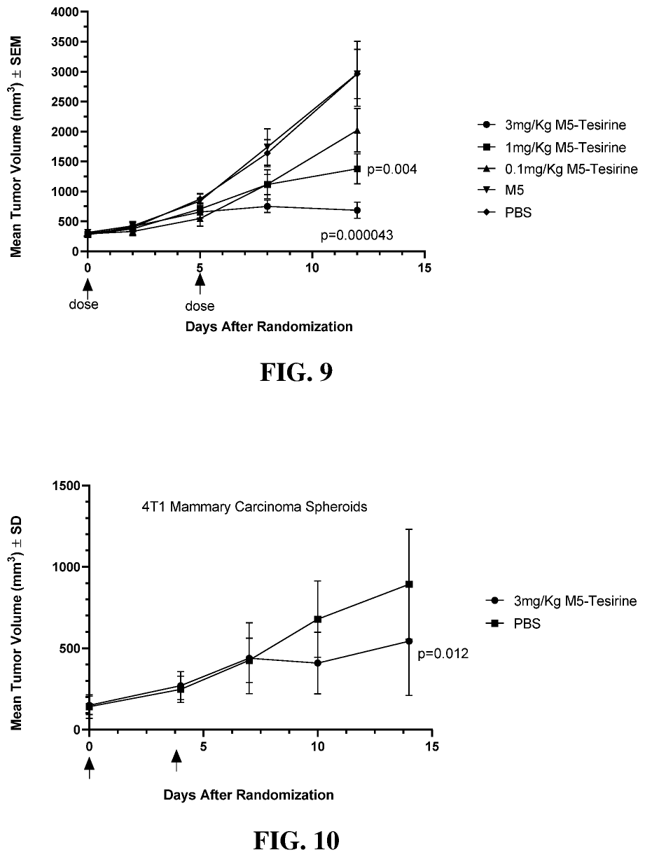

During the study, animal weights and tumor measurements were recorded twice per week. Group size consisted of 8-9 animals. Tumor volume was calculated using the formula (V=0.5326*tumor length*tumor width*tumor width). Mice were again dosed on Day 20 following the same treatment as previously used. On Day 27 Groups C, D and E were sacrificed. On Day 30, the remaining mice were sacrificed. As shown in the FIG. 9, M5-tesirine showed a statistically dose-dependent decrease in tumor volume compared to the unconjugated M5 or PBS. Unpaired T-tests were used to compare treatment arms to the PBS control.

### Example 10

### In Vivo Efficacy of M5-Tesirine ADC in a 4T.1 Spheroid Triple Negative Breast Cancer Xenograft Model

All animal work was performed in an AAALAC accredited facility, following a protocol approved by the facility's IACUC committee. Female Balb/c mice (seven weeks old, Charles River) were implanted subcutaneously into the 4^{th} mammary fat pad on the left side, with approximately sixteen 4T1 spheroids in 100 μL of PBS. Animals were monitored and when tumor volume reached 140-150 mm³, mice were randomized into two treatment groups and dosed. Group A was injected intravenously with 100 μL of PBS (n=14) and Group B (n=13) was injected intravenously with ADC (3.0 mg/kg M5/tesirine). Four days later (Day 21 following implantation), mice were again dosed using the same regimen as on Day 17.

On Day 32, the animals were sacrificed. During the study, animal weights and tumor measurements were recorded twice per week. Tumor volume was calculated using the formula (V=0.5326*tumor length*tumor width*tumor width). As shown in FIG. 10, 3.0 mg M5-tesirine (arrows indicate dosing day) was able to statistically reduce the tumor burden compared to PBS (p=0.012); Students t-test.

### Example 12

### In Vivo Efficacy of M5-ADCs in a CT26 Xenograft Model

All animal work was performed in an AAALAC accredited facility, following a protocol approved by the facility's IACUC committee. On Day 0, female Balb/c mice (seven weeks old, Charles River) were implanted subcutaneously into the 4^{th} mammary fat pad on the left side, using 500,000 CT26 cells in 100 μL PBS. Animals were monitored and when average tumor volume reached 140-160 mm³ on Day 14, mice were randomized into treatment groups and dosed intravenously (150 μL volume) using the following:

### Group A—PBS

Group B—3.0 mg/kg M5/tesirine
Group C—6.0 mg/kg M5/tesirine
Group D—9.0 mg/kg M5/tesirine
Group E—3.0 mg/kg M5/SMCC-DM1
Group F—6.0 mg/kg M5/SMCC-DM1
Group G—9.0 mg/kg M5/SMCC-DM1
Group H—3.0 mg/kg M5/MCVC-PAB-MMAE
Group I—6.0 mg/kg M5/MCVC-PAB-MMAE
Group J—9.0 mg/kg M5/MCVC-PAB-MMAE

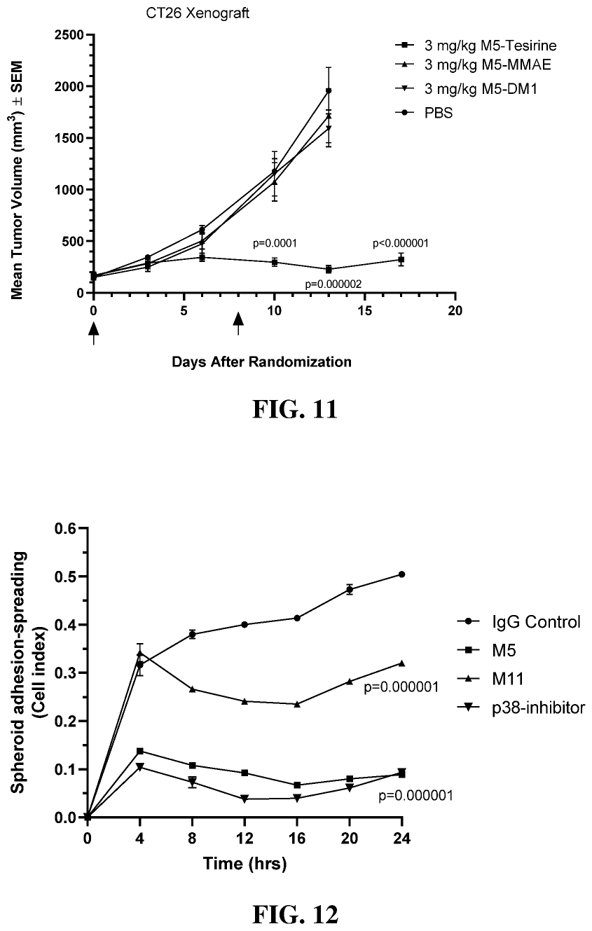

On Day 20, Groups A, B, E, H, K, and L were dosed using the same treatments as previously. Mice in Group D began to lose weight and died on Day 20, and all were sacrificed or found dead by Day 22. Mice in Group C began to lose weight Day 20 and 24, and three of them were sacrificed or found dead on Day 23. All animals were sacrificed on Day 27, except Groups B and C. Four remaining animals from Group C were harvested on Day 31, with the same tissues collected. Group B was harvested on Day 34. During the study, animal weights and tumor measurements were recorded twice per week. Group size consisted of 9-10 animals. Tumor volume was calculated using the formula (V=0.5326*tumor length*tumor width*tumor width). FIG. 11 (arrows indicate dosing day) shows that group B, 3.0 mg/kg M5-tesirine performed the best out of all of the M5 ADCs and had highly statistically significant effect (students t-test) on reducing tumor volume compared to the PBS control. Because a dose-dependent effect was not observed for the M5 ADCs on the CT-26 primary tumor, only the 3.0 mg/kg groups are shown.

### Example 13

### xCT Mabs Inhibit the Adhesion/Migration of Triple Negative Breast Cancer Cells

MDA-MB-231 spheroids were pretreated with mAbs against xCT for 60 min and then directly transferred to E-Plate (ACEA Biosciences) in the presence of RPMI containing a final 5% (vol/vol) FBS and monitored over time by an xCELLigence Real-Time Cell Analysis (RTCA) system for adhesion/spreading (over time). Cell adhesion/migration (reported as cell index) were monitored for over time (one read every 15 min) by an xCELLigence Real-Time Cell Analysis (RTCA) system. As shown in FIG. 12, treatment with mAb M5, M11, or a p38 inhibitor significantly reduced the adhesion and spreading of the spheroids compared to control (p=0.00001; multiple t-tests control PBS vs M5; PBS vs M11; multiple t-tests).

### Example 14

### xCT Mass Inhibit the Migration and Invasion of Triple Negative Breast Cancer Cells

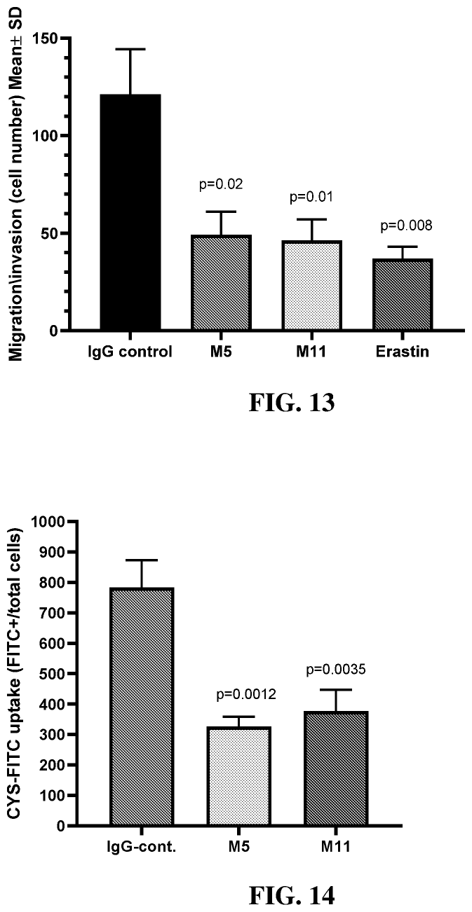

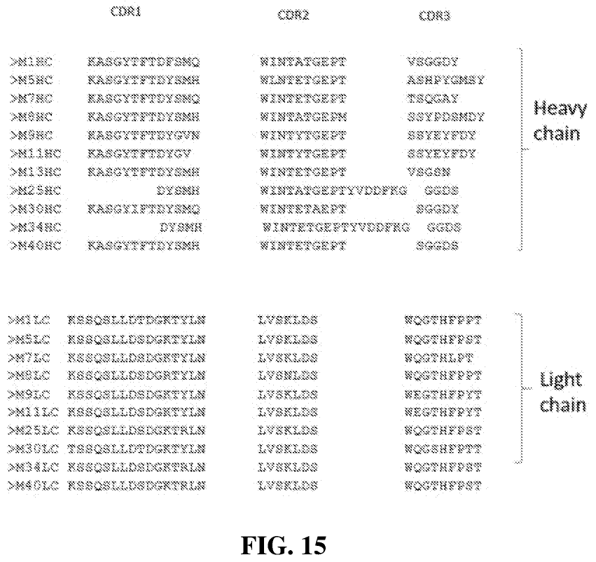

Since xCT-dependent glutamate secretion promotes cancer cell migration and invasion (Dornier, et al., Nature communications 8: 2255) we evaluated the ability of xCT mAbs in a transwell assay. Briefly MDA-MB-231 TNBC spheroids were pre-incubated with 30 μg/mL of xCT antibodies, isotype IgG control, or erastin (a known xCT inhibitor) in a serum-free medium. To prepare the spheroids, MDA-MB-231 cells suspended in complete DMEM culture medium containing 0.12% methylcellulose and 100 μg/ml collagen I were seeded in non-adherent round-bottom 96-well plates for 48 hrs. Single spheroids per well (2000 cells per spheroid) were seeded in the top chamber of a 24-transwell plate (8-μm pore size; Corning, Amsterdam, Netherlands). The bottom chambers of the trans-well plates were filled with medium supplemented with 2% FBS (ultra-low IgG) (600 μl per well) and cells were incubated at 37° C. in a 5% CO₂ atmosphere. After 48 hrs, the non-migrated cells on the top side of the filter were removed by scrubbing twice with a cotton-tipped swab. Migrated cells on the bottom side of the filter were fixed with 2.5% formaldehyde (Sigma-Aldrich) and stained with 0.2% crystal violet (Sigma-Aldrich). After washing, the migrated cells of four randomly selected fields per well were imaged using a Leica microscope (and analyzed using Fiji and ImageJ Software (Rasband, W. S., ImageJ, US National Institutes of Health). Both M5, M11 and erastin were able to significantly reduce the invasion of the MDA-MB-231 cells (FIG. 13).

### Example 15

### xCT Mabs Inihibit Cystine Uptake in TNBC MDA-MB-231 Cells

MDA-MB-231 cells starved in 1% BSA/DMEM (without L-Glutamine/Glucose) were pretreated with mAbs for 60 min followed by incubation with 200 μM of CYS-FITC in 5% FBS/DMEM for 8 hrs. Cells were fixed and stained with DAPI. Cysteine-FITC uptake was quantitated by counting cell-FITC-green/Total cells (DAPI+). Both M5 and M11 were able to statistically reduced cystine uptake by the MDA-MB-231 cells. TNBC are mean values; error bars ±SEM; (unpaired t-test control vs M5, M11).

1. A xCT antibody that specifically binds an epitope defined by the amino acid sequence of SEQ ID NO:81 or SEQ ID NO:82.

2. The antibody of claim 1, wherein the xCT antibody comprises:
(a) a heavy chain comprises a CDR1 having the amino acid sequence of SEQ ID NO:63, a CDR2 having the amino acid sequence of SEQ ID NO:64, and a CDR3 having the amino acid sequence of SEQ ID NO:65, and a light chain comprises CDR1 having the amino acid sequence of SEQ ID NO:68, a CDR2 having the amino acid sequence of SEQ ID NO:69, and a CDR3 having the amino acid sequence of SEQ ID NO:70;
(b) a heavy chain comprises a CDR1 having the amino acid sequence of SEQ ID NO:100, a CDR2 having the amino acid sequence of SEQ ID NO:101, and a CDR3 having the amino acid sequence of SEQ ID NO:102, and a light chain comprises CDR1 having the amino acid sequence of SEQ ID NO:104, a CDR2 having the amino acid sequence of SEQ ID NO:105, and a CDR3 having the amino acid sequence of SEQ ID NO:106;
(c) a heavy chain comprises a CDR1 having the amino acid sequence of SEQ ID NO:3, a CDR2 having the amino acid sequence of SEQ ID NO:4, and a CDR3 having the amino acid sequence of SEQ ID NO:5, and a light chain comprises CDR1 having the amino acid sequence of SEQ ID NO:8, a CDR2 having the amino acid sequence of SEQ ID NO:9, and a CDR3 having the amino acid sequence of SEQ ID NO:10;
(d) a heavy chain comprises a CDR1 having the amino acid sequence of SEQ ID NO:13, a CDR2 having the amino acid sequence of SEQ ID NO:14, and a CDR3 having the amino acid sequence of SEQ ID NO:15, and a light chain comprises CDR1 having the amino acid sequence of SEQ ID NO:18, a CDR2 having the amino acid sequence of SEQ ID NO:19, and a CDR3 having the amino acid sequence of SEQ ID NO:20;
(e) a heavy chain comprises a CDR1 having the amino acid sequence of SEQ ID NO:23, a CDR2 having the amino acid sequence of SEQ ID NO:24, and a CDR3 having the amino acid sequence of SEQ ID NO:25, and a light chain comprises a CDR1 having the amino acid sequence of SEQ ID NO:28, a CDR2 having the amino acid sequence of SEQ ID NO:29, and a CDR3 having the amino acid sequence of SEQ ID NO:30;
(f) a heavy chain comprises a CDR1 having the amino acid sequence of SEQ ID NO:33, a CDR2 having the amino acid sequence of SEQ ID NO:34, and a CDR3 having the amino acid sequence of SEQ ID NO:35, and a light chain comprises a CDR1 having the amino acid sequence of SEQ ID NO:38, a CDR2 having the amino acid sequence of SEQ ID NO:39, and a CDR3 having the amino acid sequence of SEQ ID NO:40;
(g) a heavy chain comprises a CDR1 having the amino acid sequence of SEQ ID NO:43, a CDR2 having the amino acid sequence of SEQ ID NO:44, and a CDR3 having the amino acid sequence of SEQ ID NO:45, and a light chain comprises a CDR1 having the amino acid sequence of SEQ ID NO:48, a CDR2 having the amino acid sequence of SEQ ID NO:49, and a CDR3 having the amino acid sequence of SEQ ID NO:50;
(h) a heavy chain comprises a CDR1 having the amino acid sequence of SEQ ID NO:73, a CDR2 having the amino acid sequence of SEQ ID NO:74, and a CDR3 having the amino acid sequence of SEQ ID NO:75, and a light chain comprises a CDR1 having the amino acid sequence of SEQ ID NO:78, a CDR2 having the amino acid sequence of SEQ ID NO:79, and a CDR3 having the amino acid sequence of SEQ ID NO:80;
(i) a heavy chain comprises a CDR1 having the amino acid sequence of SEQ ID NO:84, a CDR2 having the amino acid sequence of SEQ ID NO:85, and a CDR3 having the amino acid sequence of SEQ ID NO:86, and a light chain comprises a CDR1 having the amino acid sequence of SEQ ID NO:88, a CDR2 having the amino acid sequence of SEQ ID NO:89, and a CDR3 having the amino acid sequence of SEQ ID NO:90; or
(j) a heavy chain comprises a CDR1 having the amino acid sequence of SEQ ID NO:92, a CDR2 having the amino acid sequence of SEQ ID NO:93, and a CDR3 having the amino acid sequence of SEQ ID NO:94, and a light chain comprises a CDR1 having the amino acid sequence of SEQ ID NO:96, a CDR2 having the amino acid sequence of SEQ ID NO:97, and a CDR3 having the amino acid sequence of SEQ ID NO:98.

3. The antibody of claim 1 or 2, wherein the antibody or antibody fragment is humanized.

4. The antibody of claim 1 or 2, wherein the antibody or antibody fragment thereof is a murine/human chimeric antibody.

5. The antibody of any one of claims 1 to 4, wherein the antibody is an antibody fragment.

6. The antibody of claim 5, wherein the antibody fragment is an ScFv.

7. The antibody of claim 6, wherein the ScFv is murine, murine/human chimera, or humanized.

8. The antibody of any one of claims 1-7, wherein the antibody is an antibody conjugate having an agent conjugated to the antibody.

9. The antibody of claim 8, wherein the agent is a antimitotic cytotoxin, a DNA alkylating agent, a DNA cleaver, a DNA intercalator, a microtubule inhibitor, a topoisomerase inhibitor, chemotherapy, enzyme, radiotherapy, or a detectable label.

10. The antibody of claim 8 or 9, wherein the agent is conjugated to the antibody through a linker.

11. A method of treating brain cancer, liver cancer, pancreatic cancer, gastrointestinal cancer, lung cancer, breast cancer, cervical cancer, uterine cancer, ovarian cancers, colorectal cancer, or stomach cancer, comprising administering to a subject an effective amount of an anti-xCT antibody of any one of claims 1 to 10, or antibody fragment thereof that binds xCT.

12. The method of claim 11, further comprising contacting a biological sample from a subject comprising cancer cells with a second xCT specific antibody to identify cancer cells expressing xCT prior to administering an anti-xCT antibody of any one of claims 1 to 10.

13. The method of claim 11, wherein the antibody or antibody fragment is humanized.

14. The method of claim 11, wherein the antibody or antibody fragment thereof is a murine/human chimeric antibody.

15. The method of claim 11, wherein said antibody or antibody fragment thereof is an antibody fragment, and the antibody fragment is an ScFv.

16. The method of claim 15, wherein the ScFv is murine or humanized.

17. The method of claim 11, wherein the patient is a human or a non-human animal.

18. The method of claim 11, wherein the antibody or antibody fragment thereof is administered parenterally, intraperitoneally, intravenously, subcutaneously, orally, nasally, via inhalation or rectally.

19. The method of claim 11, wherein the antibody or antibody fragment thereof is administered intravenously at a dosage of from 0.1 mg/kg to 20 mg/kg.

20. A method of treating a subject comprising: contacting a biological sample from the subject comprising cancer cells with an xCT antibody of any one of claims 1 to 8 and detecting cancer cells expressing xCT, wherein, a subject identified as having cancer cells expressing xCT is administered an anti-cancer therapy.

21. The method of claim 20, wherein the anti-cancer therapy is an xCT antibody drug conjugate.
